SEQUENCE LISTING (86 sequences)

SEQ ID NO: 1 - protein, 12 aa, Artificial Sequence
  peptide derived from the sequence of the glucagon receptor
LVIDGLLRTGKK

SEQ ID NO: 2 - protein, 11 aa, Artificial Sequence
  peptide derived from the sequence of the glucagon receptor
AAVRCGAVGKK

SEQ ID NO: 3 - protein, 11 aa, Artificial Sequence
  peptide derived from the sequence of the glucagon receptor
FVTDEHAQGKK

SEQ ID NO: 4 - protein, 9 aa, Artificial Sequence
  peptide derived from the sequence of the glucagon receptor
LVIDGLLRT

SEQ ID NO: 5 - protein, 8 aa, Artificial Sequence
  peptide derived from the sequence of the glucagon receptor
AAVRCGAV

SEQ ID NO: 6 - protein, 8 aa, Artificial Sequence
  peptide derived from the sequence of the glucagon receptor
FVTDEHAQ

SEQ ID NO: 7 - protein, 8 aa, Artificial Sequence
  peptide derived from the sequence of the glucagon receptor
QFSSYMKA

SEQ ID NO: 8 - protein, 9 aa, Artificial Sequence
  peptide derived from the sequence of the glucagon receptor
VVKCLPENV

SEQ ID NO: 9 - protein, 8 aa, Artificial Sequence
  peptide derived from the sequence of the glucagon receptor
WFGMNDNS

SEQ ID NO: 10 - protein, 8 aa, Artificial Sequence
  peptide derived from the sequence of the glucagon receptor
FLKASRLT

SEQ ID NO: 11 - protein, 8 aa, Artificial Sequence
  peptide derived from the sequence of the glucagon receptor
QFSSYMKA

SEQ ID NO: 12 - protein, 9 aa, Artificial Sequence
  peptide derived from the sequence of the glucagon receptor
LVIDGLLRT

SEQ ID NO: 13 - protein, 8 aa, Artificial Sequence
  peptide derived from the sequence of the glucagon receptor
AAVRCGAV

SEQ ID NO: 14 - protein, 9 aa, Artificial Sequence
  peptide derived from the sequence of the glucagon receptor
VVKCLPENV

SEQ ID NO: 15 - protein, 8 aa, Artificial Sequence
  peptide derived from the sequence of the glucagon receptor
WFGMNDNS

SEQ ID NO: 16 - protein, 8 aa, Artificial Sequence
  peptide derived from the sequence of the glucagon receptor
FLKASRLT

SEQ ID NO: 17 - protein, 11 aa, Artificial Sequence
  peptide derived from the sequence of the glucagon receptor
QFSSYMKAGKK

SEQ ID NO: 18 - protein, 12 aa, Artificial Sequence
  peptide derived from the sequence of the glucagon receptor
VVKCLPENVGKK

SEQ ID NO: 19 - protein, 11 aa, Artificial Sequence
  peptide derived from the sequence of the glucagon receptor
WFGMNDNSGKK

SEQ ID NO: 20 - protein, 11 aa, Artificial Sequence
  peptide derived from the sequence of the glucagon receptor
FLKASRLTGKK

SEQ ID NO: 21 - protein, 11 aa, Artificial Sequence
  peptide derived from the sequence of the glucagon receptor
LVIDGLLRTGK

SEQ ID NO: 22 - protein, 10 aa, Artificial Sequence
  peptide derived from the sequence of the glucagon receptor
AAVRCGAVGK

SEQ ID NO: 23 - protein, 10 aa, Artificial Sequence
  peptide derived from the sequence of the glucagon receptor
FVTDEHAQGK

SEQ ID NO: 24 - protein, 10 aa, Artificial Sequence
  peptide derived from the sequence of the glucagon receptor
FLKASRLTGK

SEQ ID NO: 25 - protein, 10 aa, Artificial Sequence
  peptide derived from the sequence of the glucagon receptor
FVTDEHAAGK

SEQ ID NO: 26 - protein, 8 aa, Artificial Sequence
  peptide derived from the sequence of the glucagon receptor
FVTDEHGK

SEQ ID NO: 27 - protein, 7 aa, Artificial Sequence
  peptide derived from the sequence of the glucagon receptor
DEHAQGK

SEQ ID NO: 28 - protein, 7 aa, Artificial Sequence
  peptide derived from the sequence of the glucagon receptor
DEHAQGK

SEQ ID NO: 29 - protein, 7 aa, Artificial Sequence
  peptide derived from the sequence of the glucagon receptor
DEHAQGK

SEQ ID NO: 30 - protein, 7 aa, Artificial Sequence
  peptide derived from the sequence of the glucagon receptor
DEHAQGK

SEQ ID NO: 31 - protein, 7 aa, Artificial Sequence
  peptide derived from the sequence of the glucagon receptor
DEHAQGK

SEQ ID NO: 32 - protein, 7 aa, Artificial Sequence
  peptide derived from the sequence of the glucagon receptor
DEHAQGK

SEQ ID NO: 33 - protein, 7 aa, Artificial Sequence
  peptide derived from the sequence of the glucagon receptor
DEHAQGK

SEQ ID NO: 34 - protein, 7 aa, Artificial Sequence
  peptide derived from the sequence of the glucagon receptor
DEHAQGK

SEQ ID NO: 35 - protein, 7 aa, Artificial Sequence
  peptide derived from the sequence of the glucagon receptor
DEHAQGK

SEQ ID NO: 36 - protein, 7 aa, Artificial Sequence
  peptide derived from the sequence of the glucagon receptor
DEHAQGK

SEQ ID NO: 37 - protein, 12 aa, Artificial Sequence
  peptide derived from the sequence of the glucagon receptor
CFVTDEHAQCGK

SEQ ID NO: 38 - protein, 7 aa, Artificial Sequence
  peptide derived from the sequence of the glucagon receptor
DEHAQGK

SEQ ID NO: 39 - protein, 8 aa, Artificial Sequence
  peptide derived from the sequence of the glucagon receptor
TDEHAQGK

SEQ ID NO: 40 - protein, 7 aa, Artificial Sequence
  peptide derived from the sequence of the glucagon receptor
DEHAQGK

SEQ ID NO: 41 - protein, 8 aa, Artificial Sequence
  peptide derived from the sequence of the glucagon receptor
FVTDEHAQ

SEQ ID NO: 42 - protein, 10 aa, Artificial Sequence
  peptide derived from the sequence of the glucagon receptor
FVMDEHAQGK

SEQ ID NO: 43 - protein, 10 aa, Artificial Sequence
  peptide derived from the sequence of the glucagon receptor
FVTDEHARGK

SEQ ID NO: 44 - protein, 10 aa, Artificial Sequence
  peptide derived from the sequence of the glucagon receptor
FVMDEHARGK

SEQ ID NO: 45 - protein, 10 aa, Artificial Sequence
  peptide derived from the sequence of the glucagon receptor
FITDDQVEGK

SEQ ID NO: 46 - protein, 7 aa, Artificial Sequence
  peptide derived from the sequence of the glucagon receptor
DEHAQGK

SEQ ID NO: 47 - protein, 8 aa, Artificial Sequence
  peptide derived from the sequence of the glucagon receptor
FVTDEHAQ

SEQ ID NO: 48 - protein, 8 aa, Artificial Sequence
  peptide derived from the sequence of the glucagon receptor
FVTDEHAK

SEQ ID NO: 49 - protein, 5 aa, Artificial Sequence
  peptide derived from the sequence of the glucagon receptor
DEHAQ

SEQ ID NO: 50 - protein, 5 aa, Artificial Sequence
  peptide derived from the sequence of the glucagon receptor
DEHAQ

SEQ ID NO: 51 - protein, 5 aa, Artificial Sequence
  peptide derived from the sequence of the glucagon receptor
DEHAK

SEQ ID NO: 52 - protein, 5 aa, Artificial Sequence
  peptide derived from the sequence of the glucagon receptor
DEHAK

SEQ ID NO: 53 - protein, 5 aa, Artificial Sequence
  peptide derived from the sequence of the glucagon receptor
DEHAQ

SEQ ID NO: 54 - protein, 9 aa, Artificial Sequence
  peptide derived from the sequence of the glucagon receptor
FVTDEHAQY

SEQ ID NO: 55 - protein, 11 aa, Artificial Sequence
  peptide derived from the sequence of the glucagon receptor
DEHAQDEHAQK

SEQ ID NO: 56 - protein, 6 aa, Artificial Sequence
  peptide derived from the sequence of the glucagon receptor
DEHAQY

SEQ ID NO: 57 - protein, 8 aa, Artificial Sequence
  peptide derived from the sequence of the glucagon receptor
WDEHAQGK

SEQ ID NO: 58 - protein, 9 aa, Artificial Sequence
  peptide derived from the sequence of the glucagon receptor
DEHADEHAK

SEQ ID NO: 59 - protein, 5 aa, Artificial Sequence
  peptide derived from the sequence of the glucagon receptor
DEHAQ

SEQ ID NO: 60 - protein, 5 aa, Artificial Sequence
  peptide derived from the sequence of the glucagon receptor
DEHAK

SEQ ID NO: 61 - protein, 8 aa, Artificial Sequence
  cyclic peptide derived from the sequence of the glucagon receptor
FVXDEHAQ

SEQ ID NO: 62 - protein, 29 aa, Artificial Sequence
  peptide, mammalian glucagon
HSQGTFTSDYSKYLDSRRAQDFVQWLMNT

SEQ ID NO: 63 - protein, 28 aa, Artificial Sequence
  peptide, variant of mammalian glucagon (DesHis1, Glu9) Glucagon Amide
SQGTFTSEYSKYLDSRRAQDFVQWLMNT

SEQ ID NO: 64 - protein, 30 aa, Artificial Sequence
  peptide, fragment of Glucagon Like Peptide-1 (GLP-1)
HAEGTFTSDVSSYLEGQAAKEFIAWLVKGR

SEQ ID NO: 65 - protein, 8 aa, Artificial Sequence
  peptide derived from the sequence of the glucagon receptor
QFSSYMKA

SEQ ID NO: 66 - protein, 9 aa, Artificial Sequence
  peptide derived from the sequence of the glucagon receptor
LVIDGLLRT

SEQ ID NO: 67 - protein, 8 aa, Artificial Sequence
  peptide derived from the sequence of the glucagon receptor
AAVRCGAV

SEQ ID NO: 68 - protein, 9 aa, Artificial Sequence
  peptide derived from the sequence of the glucagon receptor
VVKCLPENV

SEQ ID NO: 69 - protein, 8 aa, Artificial Sequence
  peptide derived from the sequence of the glucagon receptor
WFGMNDNS

SEQ ID NO: 70 - protein, 8 aa, Artificial Sequence
  peptide derived from the sequence of the glucagon receptor
FLKASRLT

SEQ ID NO: 71 - protein, 5 aa, Artificial Sequence
  peptide derived from the sequence of the glucagon receptor
DEHAQ

SEQ ID NO: 72 - protein, 10 aa, Artificial Sequence
  peptide derived from the sequence of the glucagon receptor
CFVTDEHAQC

SEQ ID NO: 73 - protein, 6 aa, Artificial Sequence
  peptide derived from the sequence of the glucagon receptor
TDEHAQ

SEQ ID NO: 74 - protein, 8 aa, Artificial Sequence
  peptide derived from the sequence of the glucagon receptor
FVMDEHAQ

SEQ ID NO: 75 - protein, 8 aa, Artificial Sequence
  peptide derived from the sequence of the glucagon receptor
FVTDEHAR

SEQ ID NO: 76 - protein, 8 aa, Artificial Sequence
  peptide derived from the sequence of the glucagon receptor
FVMDEHAR

SEQ ID NO: 77 - protein, 8 aa, Artificial Sequence
  peptide derived from the sequence of the glucagon receptor
FITDDQVE

SEQ ID NO: 78 - protein, 5 aa, Artificial Sequence
  peptide derived from the sequence of the glucagon receptor
DEHAQ

SEQ ID NO: 79 - protein, 8 aa, Artificial Sequence
  peptide derived from the sequence of the glucagon receptor
FVTDEHAK

SEQ ID NO: 80 - protein, 5 aa, Artificial Sequence
  peptide derived from the sequence of the glucagon receptor
DEHAK

SEQ ID NO: 81 - protein, 5 aa, Artificial Sequence
  peptide derived from the sequence of the glucagon receptor
DEHAK

SEQ ID NO: 82 - protein, 9 aa, Artificial Sequence
  peptide derived from the sequence of the glucagon receptor
FVTDEHAQY

SEQ ID NO: 83 - protein, 10 aa, Artificial Sequence
  peptide derived from the sequence of the glucagon receptor
DEHAQDEHAQ

SEQ ID NO: 84 - protein, 6 aa, Artificial Sequence
  peptide derived from the sequence of the glucagon receptor
DEHAQY

SEQ ID NO: 85 - protein, 477 aa, Homo sapiens
MPPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 86 - protein, 8 aa, Artificial Sequence
  peptide derived from the sequence of the glucagon receptor
DEHADEHA

(19) United States
(12) Patent Application Publication
(10) Pub. No.: US 2005/0124550 A1
(43) Pub. Date: Jun. 9, 2005

(54) Compounds that modulate the glucagon response and uses thereof

(76) Inventor: Krishna G. Peri, Quebec (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/871,885

(22) Filed: Jun. 18, 2004

Related U.S. Application Data
(60) Provisional application No. 60/479,692, filed on Jun. 18, 2003.

(51) Int. Cl. A61K 38/06; A61K 38/08; C07K 7/06

(52) U.S. Cl. 514/16; 514/17; 530/330; 530/329

(57) ABSTRACT

Peptides that modulate the glucagon response in a mammal are provided. The peptides comprise an amino acid sequence of between about 5 and about 10 amino acids in length that corresponds to the sequence of an extracellular membrane insertion region of a mammalian glucagon receptor, wherein at least one amino acid of the peptide has a D-configuration. Methods of preparing the peptides and the use of the peptides in the amelioration, treatment and/or prevention of glucagon-mediated conditions and diseases such as hyperglycemia, diabetes and obesity are also provided.

### FIELD OF THE INVENTION

The present invention pertains to the field of therapeutics, and in particular to therapeutic compounds for the treatment and/or prevention of glucagon-mediated diseases such as hyperglycemia and diabetes.

### BACKGROUND

Native glucagon is a 29 amino acid peptide, the key physiological action of which is the regulation of blood glucose levels through enhanced synthesis and mobilization of glucose in the liver.

Glucagon generally functions as a counter-regulatory hormone, opposing the actions of insulin, to maintain the level of blood glucose, particularly in instances of hypoglycemia. However, in some patients with Type 1 or Type 2 diabetes, absolute or relative elevated glucagon levels have been shown to contribute to the hyperglycemic state. Both in healthy control animals as well as in animal models of Type 1 and Type 2 diabetes, removal of circulating glucagon with selective and specific antibodies has resulted in reduction of the glycemic level (Brand et al., Diabetologia 37, 985 (1994); Diabetes 43, [suppl 1], 172A (1994); Am. J. Physiol. 269, E469-E477 (1995); Diabetes 44 [suppl 1], 134A (1995); Diabetes 45, 1076 (1996)). These studies suggest that glucagon antagonism could be a useful in glycemic control in the treatment of diabetes.

Glucagon exerts its action by binding to and activating its receptor, which is part of the glucagon-secretin branch of the 7-transmembrane G-protein coupled receptor family (Jelinek et al., Science 259, 1614, (1993)). The receptor functions by an activation of the adenylyl cyclase resulting in increased cAMP levels.

Other members of the glucagon-secretin branch of the 7-transmembrane G-protein coupled receptor family include the prostaglandin receptor, PGF_{2[alpha]} and PGE₂. Peptide antagonists that inhibit the function of these receptor by binding to an intracellular portion, an extracellular portion or to a juxtamembrane extracellular structural element have been described (see, U.S. Pat. Nos. 5,955,575 and 6,300,312; Canadian Patent Application Nos 2,342,960 and 2,396,739).

Inhibitors of the glucagon receptor have been described and are generally based on the amino acid sequence of glucagon. Several analogues in which one or more amino acids were either deleted or substituted to produce potent antagonists of glucagon receptor have been described, for example, [des His¹] [Glu⁹]-glucagon amide (Unson et al., 1989. Peptides 10, 1171; Post et al., 1993. Proc. Natl. Acad. Sci. USA 90, 1662), DesHis¹, Phe⁶ [Glu⁹]-glucagon amide (Azizh et al. 1995. Bioorg. & Med. Chem. Lett. 16, 1849) and Nle⁹, Ala^{11,16}-glucagon amide (Unson et al. 1994. J. Biol. Chem. 269(17), 12548). Other analogues include substitutions at positions 4 (Ahn J M et al. 2001. J. Pept. Res. 58(2):151-8), 1 (Dharanipragada, R. et al. 1993. Int. J. Pept. Res. 42(1): 68-77) and 4, 5, 12, 17 and 18 in the glucagon sequence (Gysin B et al. 1986. Biochemistry. 25(25):8278-84). Peptide antagonists of the glucagon receptor that were obtained through random screening by peptide display technologies and which are not based on glucagon sequence have also been described (see, published International Patent Application WO 01/83527).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide compounds that modulate the glucagon response and uses thereof. In accordance with one aspect of the present invention, there is provided a peptide comprising an amino acid between about 5 and about 10 amino acids in length, said amino acid sequence corresponding to a sequence of an extracellular membrane insertion region of a mammalian glucagon receptor and comprising at least one D-amino acid, wherein said peptide is capable of modulating the glucagon response in a mammal.

In accordance with another aspect of the invention, there is provided a pharmaceutical composition comprising one or more peptide according to claim 1 and a pharmaceutically acceptable diluent, carrier or excipient.

In accordance with another aspect of the invention, there is provided a method of modulating cAMP levels in a mammal comprising administering to said mammal an effective amount of one or more peptide of the invention.

In accordance with another aspect of the invention, there is provided a method of modulating blood glucose levels in a mammal comprising administering to said mammal an effective amount of one or more peptide of the invention.

In accordance with another aspect of the invention, there is provided a method of modulating the glucagon response in a mammal comprising administering to said mammal an effective amount of one or more peptide of the invention.

In accordance with another aspect of the invention, there is provided a method of treating or preventing a glucagon-mediated disease, disorder or condition in a mammal comprising administering to said mammal an effective amount of one or more peptide of the invention.

In accordance with another aspect of the invention, there is provided a method of determining the ability of a peptide to modulate the glucagon response, said method comprising the steps of:
a) contacting cells or tissue responsive to glucagon with a candidate peptide and a known glucagon antagonist;
b) after an appropriate period of time, contacting said cells or tissue with glucagon to elicit a glucagon response; and
c) measuring one or more biochemical consequences of the cell before the addition of glucagon and at appropriate time intervals after the addition of glucagon, wherein said biochemical consequence is cAMP or glucose levels;
   wherein a change in the measured biochemical consequence compared to a negative control indicates that said peptide is capable of modulating the glucagon response.

In accordance with another aspect of the invention, there is provided a kit comprising:
a) one or more peptides of the invention; and
b) optionally instructions for use.

### BRIEF DESCRIPTION OF THE FIGURES

FIG. 1 depicts the amino acid sequence of the human glucagon receptor (Accession No. NP 000151) (SEQ ID NO:85);

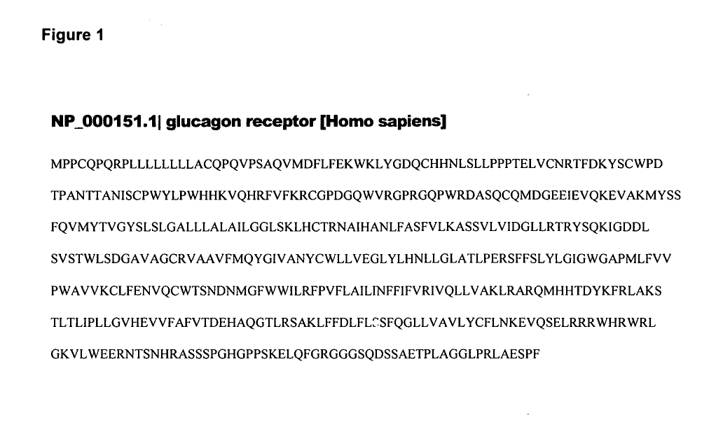

FIG. 2 depicts the effect of Peptide Nos. 1, 2 and 3 (1 mg/kg iv) and a known glucagon receptor antagonist, desHis¹, glu⁹ glucagon (1-29) amide (10 μg/rat iv) on glucagon (1-29) amide (4 μg/rat iv)-induced increase in blood glucose levels in rats in terms of % increase in glucose levels compared to the basal values (n=4-7 rats/group);

FIG. 3 depicts the area under the curves (‘AUC’) (30-60 min) in FIG. 2, calculated using Graphpad Prism (Graphpad Software version 3.03);

FIG. 4 depicts the percent increase in average (of 30, 45 and 60 min values from FIG. 2) glucose levels from the base line;

FIG. 5 depicts the effects of 0.1 μM each of various derivatives of Peptide No. 3 on glucagon (0.1 μM)-induced cAMP levels in rat liver primary hepatocytes. Data are means±SEM; n is number of independent experiments and shown at the top of the bars;

FIG. 6 presents dose-response curves of two derivatives of Peptide No. 3 on cAMP production induced by 10⁻⁷ M glucagon in isolated hepatocytes. Data are transformed as % maximal response and shown as means±SEM; EC₅₀ and % inhibition are shown in the legend; n is two independent experiments in which each treatment was performed in triplicate wells;

FIG. 7 depicts the displacement of radiolabeled glucagon (¹²⁵I) with glucagon, [des-His¹, Glu⁹] glucagon amide, Peptide Nos. 47 and 49. Data (mean±SEM) represent the average of 2 experiments done in triplicate and are presented as IC₅₀ and percent displacement of bound radiolabeled glucagon;

FIG. 8 depicts the displacement of radiolabeled ¹²⁵I-Peptide No. 54 with glucagon, Peptide Nos. 47 and 49. Data.(mean±SEM) represent the average of 2 experiments done in triplicate and are presented as IC₅₀ and percent displacement of bound radiolabeled peptide;

FIG. 9 depicts the effects of various peptides (300 μg/kg; sc) on blood glucose (A) and cAMP (B) level increase following portal vein injections of glucagon (12 μg/kg). Data (mean±SEM) represent the average of 3-6 experiments;

FIG. 10 demonstrates the glucagon-induced blood glucose increase (% over basal level) in the presence or absence of intravenous administration of selected peptides (1 mg/kg) and desHis¹ glu⁹ glucagon amide (10 μg/rat) in Sprague Dawley rats that fasted for 4 hours (A). Glucagon was injected intravenously 2-5 minutes after injections of the peptides. Area under the curves (AUC) were compared (B);

FIG. 11 depicts the dose-dependent effects of (A) Peptide No. 47; (C) Peptide No. 49 (20-400 μg/kg sc); (E) [Des-His¹, Glu⁹] glucagon on blood glucose levels in 4 hrs fasted CD-1 mice; corresponding AUC of blood glucose are shown in (B), (D) and (F). (Data are mean±SEM expressed as delta glucose; n is presented on top of AUC bars; *P<0.05);

FIG. 12 depicts the effects of des-His¹, Glu⁹ glucagon and selected peptides on blood glucose levels in 4 hrs fasted Sprague-Dawley rats. (Data are mean±SEM of AUC for 60 min; 400 μg/kg sc);

FIG. 13 depicts the effects of Peptide Nos. 47 and 49 and GLP-1 on Streptotozocin-induced diabetic CD-1 mice blood glucose level (A) and the corresponding AUC for 2 h (B). (Data are mean±SEM of AUC for 60 min; peptide dose—400 μg/kg sc; n=7). The experiment was performed 24 hours post-streptozocin injections when blood glucose levels have reached 20-26 mmol/L; and

FIG. 14 depicts the dose-dependent effects of Peptide No. 23 (1, 2, 5, 10, 25, 50, and 100 μg/kg) on stress-induced glucose increase in fa/fa rats. (A) data expressed as percent increase from baseline; (B). the relative AUC at the different doses plotted to provide an ED₅₀ value. Data are mean±SEM, numbers of rats (Saline: 8; others: 4 each).

### DETAILED DESCRIPTION OF THE INVENTION

Definitions

Unless defined otherwise, all technical and scientific terms used herein have the same meaning as commonly understood by one of ordinary skill in the art to which this invention pertains.

The term “peptide,” as used herein, refers to a sequence of amino acid residues linked together by peptide bonds or by modified peptide bonds. The term “peptide” is intended to encompass peptide analogues, peptide derivatives, peptidomimetics and peptide variants.

The term “peptide analogue,” as used herein, refers to a sequence of amino acid residues comprising one or more non-naturally occurring amino acids. Examples of non-naturally occurring amino acids include, but are not limited to, D-amino acids (i.e. an amino acid of an opposite chirality to the naturally occurring form), N-α-methyl amino acids, C-α-methyl amino acids, β-methyl amino acids, D or L-β-amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine D- or L-p-methoxybiphenylalanine, methionine sulphoxide (MSO) and homoarginine (Har). Other examples include substituted β-alanine (β-Ala), wherein one or more substituents of β-alanine are selected from arylsulfonyl, alkoxycarbonyl. Non-limiting examples of arylsulfonyl groups are benzenesulfonyl and 2-naphthalene sulfonyl. A non-limiting example of alkoxycarbonyl is t-butoxycarbonyl. Further examples include D- or L-2-indole(alkyl)alanines and D- or L-alkylalanines, wherein alkyl is substituted or unsubstituted methyl, ethyl, propyl, hexyl, butyl, pentyl, hexyl, octyl, isopropyl, iso-butyl, or iso-pentyl, and phosphono- or sulphated (e.g. —SO₃H) non-carboxylate amino acids.

The term “peptide derivative,” as used herein, refers to a sequence of amino acid residues comprising additional chemical or biochemical moieties not normally a part of a naturally-occurring peptide. Peptide derivatives include peptides in which the amino-terminus and/or the carboxy-terminus and/or one or more amino acid side chain has been derivatised with a suitable chemical substituent group, as well as cyclic peptides, dual peptides, multimers of the peptides, peptides fused to other proteins or carriers, glycosylated peptides, phosphorylated peptides, peptides conjugated to lipophilic moieties (for example, octyl, caproyl, lauryl, stearoyl moieties) and peptides conjugated to an antibody or other biological ligand. Examples of chemical substituent groups that may be used to derivatise a peptide include, but are not limited to, alkyl, alkenyl, alkynyl, aryl, aralkyl, heteroalkyl, a heterocyclic ring, a heteroaromatic ring, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene, and NZ₁Z₂ where Z₁ and Z₂ are independently hydrogen, alkyl, aryl, or aralkyl, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides.

The term “peptidomimetic,” as used herein, refers to a compound that is structurally similar to a peptide of naturally-occurring amino acids and contains chemical moieties that mimic the function of the peptide. For example, if a peptide contains two charged chemical moieties having functional activity, a mimetic places two charged chemical moieties in a spatial orientation and constrained structure so that the charged chemical function is maintained in three-dimensional space. The term peptidomimetic thus is intended to include isosteres. The term “isostere,” as used herein, refers to a chemical structure that can be substituted for a peptide because the steric conformation of the chemical structure is similar, for example, the structure fits a binding site specific for the peptide. Examples of peptidomimetics include peptides comprising one or more backbone modifications (i.e. amide bond mimetics), which are well known in the art. Examples of amide bond mimetics include, but are not limited to, —CH₂NH—, —CH₂S—, —CH₂CH₂—, —CH═CH— (cis and trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)). Other examples of peptidomimetics include peptides substituted with one or more benzodiazepine molecules (see, for example, James, G. L. et al. (1993) Science 260:1937-1942) and peptides comprising backbones crosslinked to form lactams or other cyclic structures.

The term “variant peptide,” as used herein, refers to a sequence of amino acid residues in which one or more amino acid residue has been deleted, added or substituted in comparison to the amino acid sequence of the extracellular membrane insertion region of a glucagon receptor to which the peptide corresponds. Typically, when a variant contains one or more amino acid substitutions they are “conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

The term “naturally-occurring,” as used herein with reference to an object, such as a protein, polypeptide or peptide, indicates that the object can be found in nature. For example, a protein, polypeptide or peptide that is present in an organism (including viruses) or that can be isolated from a source in nature and which has not been intentionally modified by man in the laboratory is naturally-occurring.

The term “alkyl,” as used herein, refers to a straight chain or branched hydrocarbon of one to ten carbon atoms or a cyclic hydrocarbon group of three to ten carbon atoms. Said alkyl group is optionally substituted with one or more substituents independently selected from the group of: alkyl, alkenyl, alkynyl, aryl, heteroalkyl, a heterocyclic ring, a heteroaromatic ring, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene and NZ₁Z₂ where Z₁ and Z₂ are independently hydrogen, alkyl, aryl, and aralkyl. This term is exemplified by such groups as methyl, ethyl, n-propyl, i-propyl, n-butyl, t-butyl, 1-butyl (or 2-methylpropyl), cyclopropylmethyl, i-amyl, n-amyl, hexyl, cyclopropyl, cyclobutyl, cyclopentyl, and the like.

The term “alkenyl” refers to a straight chain or branched hydrocarbon of two to ten carbon atoms having at least one carbon to carbon double bond. Said alkenyl group can be optionally substituted with one or more substituents as defined above. Exemplary groups include allyl and vinyl.

The term “alkynyl” refers to a straight chain or branched hydrocarbon of two to ten carbon atoms having at least one carbon to carbon triple bond. Said alkynyl group can be optionally substituted with one or more substituents as defined above. Exemplary groups include ethynyl and propargyl.

The term “heteroalkyl,” as used herein, refers to an alkyl group of 1 to 10 carbon atoms, wherein at least one carbon is replaced with a hetero atom, such as N, O or S.

The term “aryl” (or “Ar”), as used herein, refers to an aromatic carbocyclic group containing about 6 to about 10 carbon atoms or multiple condensed rings in which at least one ring is aromatic carbocyclic group containing 6 to about 10 carbon atoms. Said aryl or Ar group can be optionally substituted with one or more substituents as defined above. Exemplary aryl groups include phenyl, tolyl, xylyl, biphenyl, naphthyl, 1,2,3,4-tetrahydronaphthyl, anthryl, phenanthryl, 9-fluorenyl, and the like.

The term “aralkyl,” as used herein, refers to a straight or branched chain alkyl, alkenyl or alkynyl group, wherein at least one of the hydrogen atoms is replaced with an aryl group, wherein the aryl group can be optionally substituted with one or more substituents as defined above. Exemplary aralkyl group include benzyl, 4-phenylbutyl, 3,3-diphenylpropyl etc.

The term “alkoxy,” as used herein, refers to RO—, wherein R is alkyl, alkenyl or alkynyl in which the alkyl, alkenyl and alkynyl groups are as previously described. Exemplary alkoxy goups include methoxy, ethoxy, n-propoxy, I-propoxy, n-butoxy, and heptoxy.

The term “aryloxy” as used herein, refers to an “aryl-O—” group in which the aryl group is as previously described. Exemplary aryloxy goups include phenoxy and naphthoxy.

The term “alkylthio,” as used herein, refers to RS—, wherein R is alkyl, alkenyl or alkynyl in which the alkyl, alkenyl and alkynyl groups are as previously described. Exemplary alkylthio goups include methylthio, ethylthio, I-propylthio and hepthylthio.

The term “arylthio,” as used herein, refers to an “aryl-S—” group in which the aryl group is as previously described. Exemplary arylthio goups include phenylthio and naphthylthio.

The term “aralkyloxy,” as used herein, refers to an “aralkyl-O—” group in which the aralkyl group is as previously described. Exemplary aralkyloxy goups include benzyloxy.

The term “aralkylthio,” as used herein, refers to an “aralkyl-S—” group in which the aralkyl group is as previously described. Exemplary aralkylthio goups include benzylthio.

The term “dialkylamino,” as used herein, refers to an —NZ₁Z₂ group wherein Z₁ and Z₂ are independently selected from alkyl, alkenyl or alkynyl, wherein alkyl, alkenyl and alkynyl are as previously described. Exemplary dialkylamino groups include ethylmethylamino, dimethylamino and diethylamino.

The term “alkoxycarbonyl,” as used herein, refers to R—O—CO—, wherein R is alkyl, alkenyl or alkynyl, wherein alkyl, alkenyl and alkynyl are as previously described. Exemplary alkoxycarbonyl groups include methoxy-carbonyl and ethoxy-carbonyl.

The term “aryloxycarbonyl,” as used herein, refers to an “aryl-O—CO—”, wherein aryl is as defined previously. Exemplary aryloxycarbonyl groups include phenoxy-carbonyl and naphtoxy-carbonyl.

The term “aralkoxycarbonyl,” as used herein, refers to an “aralkyl-O—CO—,” wherein aralkyl is as defined previously. Exemplary aralkoxycarbonyl groups include benzyloxycarbonyl.

The term “heterocyclic,” as used herein, refers to a saturated, unsaturated, or aromatic carbocyclic group having a single ring having 3 to 10 carbons (e.g., morpholino, pyridyl or furyl) or multiple condensed rings (e.g., naphthpyridyl, quinoxalyl, quinolinyl, indolizinyl, indanyl or benzo[b]thienyl) and having at least one hetero atom, such as N, O or S, within the ring.

The term “heteroaromatic,” as used herein, refers to a heterocycle in which at least one heterocyclic ring is aromatic.

The term “acyl” as used herein, refers to RC(O)—, wherein R is alkyl, alkenyl, alkynyl, heteroalkyl, a heterocyclic ring, or a heteroaromatic ring, wherein alkyl, alkenyl, alkynyl, heteroalkyl, heterocyclic, and heteroaromatic are as defined previously.

The term “aroyl” as used herein, refers to an ArC(O)— group, wherein Ar is as defined previously.

The term “carboxy” as used herein, refers to ROC(O)—, wherein R is H, alkyl, alkenyl or alkynyl, and wherein alkyl, alkenyl or alkynyl are as defined previously.

The term “carbamoyl,” as used herein, refers to a H₂N—CO— group.

The term “alkylcarbamoyl,” as used herein, refers to an “Z₁Z₂N—CO—” group wherein one of the Z₁ and Z₂ is hydrogen and the other of Z₁ and Z₂ is independently selected from alkyl, alkenyl or alkynyl and wherein alkyl, alkenyl and alkynyl are as defined previously.

The term “dialkylcarbamoyl,” as used herein, refers to a “Z₁Z₂N—CO—” group wherein Z₁ and Z₂ are independently selected from alkyl, alkenyl or alkynyl and wherein alkyl, alkenyl and alkynyl are as defined previously.

The term “acylamino”, as used herein, refers to an “acyl-NH—” group, wherein acyl is as defined previously.

The term “halo” as used herein, refers to fluoro, chloro, bromo or iodo. In one embodiment, “halo” refers to fluoro, chloro or bromo.

Naturally-occurring amino acids are identified throughout by the conventional three-letter or one-letter abbreviations indicated below, which are as generally accepted in the peptide art and are recommended by the IUPAC-IUB commission in biochemical nomenclature:

TABLE 1
Amino acid codes

| | 3-letter | 1-letter |
|---|---|---|
| Name | code | code |
| Alanine | Ala | A |
| Arginine | Arg | R |
| Asparagine | Asn | N |
| Aspartic | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The peptide sequences set out herein are written according to the generally accepted convention whereby the N-terminal amino acid is on the left and the C-terminal amino acid is on the right. By convention, L-amino acids are represented by upper case letters and D-amino acids by lower case letters.

Peptides of the Invention

The present invention provides for peptides that modulate the glucagon response in a mammal. The peptides of the present invention include peptide analogues, peptide derivatives, peptidomimetics, peptide variants and combinations thereof. Such compounds are well known in the art and may have advantages over naturally occurring peptides, including, for example, greater chemical stability, increased resistance to proteolytic degradation, enhanced pharmacological properties (such as, half-life, absorption, potency and efficacy), altered specificity (for example, a broad-spectrum of biological activities) and/or reduced antigenicity.

In accordance with the present invention, the peptides comprise an amino acid sequence of about 5 to about 10 amino acids in length that corresponds wholly or in part to the sequence of an extracellular membrane insertion region of a glucagon receptor, wherein at least one amino acid of the peptide has a D-configuration. By “wholly or in part” it is meant that between one amino acid and all the amino acids of the sequence of 5 to 10 amino acids comprised by the peptide correspond to an extracellular membrane insertion region of a glucagon receptor. In one embodiment, between two amino acids and all the amino acids of the sequence of 5 to 10 amino acids comprised by the peptide correspond to the sequence of an extracellular membrane insertion region of a glucagon receptor. In another embodiment, between three amino acids and all the amino acids of the sequence of 5 to 10 amino acids comprised by the peptide correspond to an extracellular membrane insertion region of a glucagon receptor. In a further embodiment, between four amino acids and all the amino acids of the sequence of 5 to 10 amino acids comprised by the peptide correspond to an extracellular membrane insertion region of a glucagon receptor. In another embodiment, between five amino acids and all the amino acids of the sequence of 5 to 10 amino acids comprised by the peptide correspond to an extracellular membrane insertion region of a glucagon receptor. In other embodiments, the peptides comprise a sequence of at least five, at least six, at least seven, at least 8 and at least 9 amino acids corresponding to an extracellular membrane insertion region of a glucagon receptor.

The sequence of the peptide may run in the same direction as that of the corresponding sequence in the glucagon receptor (i.e. the N-terminus of the peptide corresponds to the N-terminal end of the corresponding amino acid sequence in the receptor), or the sequence of the peptide may be inverted (i.e. the N-terminus of the peptide corresponds to the C-terminal end of the corresponding amino acid sequence in the receptor). For example, for an extracellular membrane insertion region sequence of: NH₂—VAGCRVAA-CO₂H, the sequence of an inverted (“retro”) peptide corresponding to this region would be: NH₂-AAVRCGAV—CO₂H.

As is known in the art, mammalian glucagon receptors comprise 7 transmembrane domains (domains 1 through 7) linked by extracellular and intracellular loops. The peptides of the present invention comprise an amino acid sequence that corresponds wholly or in part to one of the 7 extracellular membrane insertion regions of the protein, i.e. where an extracellular loop joins a transmembrane domain. These extracellular membrane insertion regions occur both where an extracellular loop enters the membrane to become a transmembrane domain and where a transmembrane domain exits the membrane into the extracellular space to create an extracellular loop. A worker skilled in the art will appreciate that the transmembrane domains of receptor proteins are not rigidly defined but exhibit a certain fluidity and, therefore, that the membrane insertion region is not a static point corresponding to a particular amino acid, but rather is a dynamic region comprising several amino acids, typically about 10 amino acids. The peptides of the invention comprise an amino acid sequence that corresponds to a region of the receptor that, in general, is partially within the membrane and partially in the extracellular space, but which may be at times situated either entirely within the membrane or entirely in the extracellular space.

Candidate peptides can be selected based on the amino acid sequences of a mammalian glucagon receptor and tested according to standard methods, such as those described herein, for their ability to modulate the glucagon response. The amino acid sequences of various mammalian glucagon receptors are known in the art, for example, the sequences for the human, mouse and rat glucagon receptors are available from GenBank (Accession Nos. NP 000151 [Homo sapiens] (SEQ ID NO:85); NP 742089 and NP 742088 [Rattus norvegicus]; NP 032127 [Mus musculus]). The predicted transmembrane domains of a number of glucagon receptors have already been identified and thus the extracellular membrane insertion regions can be readily determined. Alternatively, the transmembrane domains of the selected receptor can be predicted using standard techniques. Methods of identifying putative transmembrane domains are known in the art and include, for example, hydropathy plots such as those of Kyte-Doolittle, Hopp-Wood and Eisenberg. Alternatively, transmembrane domains may be determined by computer modeling using the structure of a known receptor, such as rhodopsin, as a basis.

In one embodiment of the present invention, candidate peptides are selected that comprise an amino acid sequence between about 5 to about 10 amino acids in length that corresponds to wholly or in part to the sequence of an extracellular membrane insertion region of a glucagon receptor. In another embodiment, candidate peptides comprise about 7 to about 9 amino acids corresponding to the sequence of an extracellular membrane insertion region of a glucagon receptor. In a further embodiment, the glucagon receptor is a human glucagon receptor. In another embodiment, the glucagon receptor is a human glucagon receptor having a sequence substantially identical to that set forth in SEQ ID NO:85.

As indicated above, the peptides of the present invention include peptide variants. Thus, once the sequence of an extracellular membrane insertion region of the receptor has been determined and selected as the basis for the design of a candidate peptide, a peptide comprising a variant of this sequence can be designed. In one embodiment of the invention, a peptide variant comprises an amino acid sequence between about 5 and about 10 amino acids in length that corresponds to the sequence of an extracellular membrane insertion region of a glucagon receptor with one or more amino acid deletion, insertion or substitution. In accordance with one embodiment of the invention, a variant peptide has at least about 70% identity to the corresponding extracellular membrane insertion region sequence. In another embodiment, a variant peptide has at least about 80% identity to the corresponding extracellular membrane insertion region sequence. In another embodiment, a variant peptide has at least about 90% identity to the corresponding extracellular membrane insertion region sequence.

In an alternative embodiment of the invention, a variant peptide comprises an amino acid sequence between about 5 and about 10 amino acids in length that corresponds to the sequence of an extracellular membrane insertion region of a glucagon receptor with between one and five amino acid deletions, insertions or substitutions. In another embodiment, a variant peptide comprises an amino acid sequence that has five or less deletions, insertions or substitutions when compared to the sequence of an extracellular membrane insertion region of a glucagon receptor. In another embodiment, a variant peptide comprises an amino acid sequence that has four or less deletions, insertions or substitutions when compared to the sequence of an extracellular membrane insertion region of a glucagon receptor. In a further embodiment, a variant peptide comprises an amino acid sequence that has three or less deletions, insertions or substitutions when compared to the sequence of an extracellular membrane insertion region of a glucagon receptor. In another embodiment, a variant peptide comprises an amino acid sequence that has two or less deletions, insertions or substitutions when compared to the sequence of an extracellular membrane insertion region of a glucagon receptor.

The peptides of the invention can be peptide analogues. As is known in the art, substitution of all L-amino acids within the peptide with D-amino acids results in either an “inverso” peptide, or in a “retro-inverso” peptide (see Goodman et al. “Perspectives in Peptide Chemistry” pp. 283-294 (1981); U.S. Pat. No. 4,522,752), both of which are considered to be peptide analogues in the context of the present invention. An “inverso” peptide is one in which all L-amino acids of a sequence have been replaced with D-amino acids, and a “retro-inverso” peptide is one in which the sequence of the amino acids has been reversed (“retro”) and all L-amino acids have been replaced with D-amino acids. For example, if the parent peptide is Thr-Ala-Tyr, the retro form is Tyr-Ala-Thr, the inverso form is thr-ala-tyr, and the retro-inverso form is tyr-ala-thr (lower case letters indicating D-amino acids). Compared to the parent peptide, a retro-inverso peptide has a reversed backbone while retaining substantially the original spatial conformation of the side chains, resulting in an isomer with a topology that closely resembles the parent peptide.

In one embodiment of the present invention, the peptides comprise an amino acid sequence that corresponds wholly or in part to the extracellular membrane insertion region of transmembrane domains 2, 4 or 6 of a glucagon receptor and have a sequence that directly corresponds to the sequence of that region. In another embodiment, the peptides comprise an amino acid sequence that corresponds wholly or in part to the extracellular membrane insertion region of transmembrane domains 1, 3, 5 or 7 of a glucagon receptor and have an inverted (“retro”) sequence relative to the sequence of that region. In another embodiment, the peptides are “inverso” peptides having a sequence that corresponds wholly or in part to the extracellular membrane insertion region of transmembrane domains 2, 4 or 6 of a glucagon receptor. In a further embodiment, the peptides are “retro-inverso” peptides corresponding to the extracellular membrane insertion region of transmembrane domains 1, 3, 5 or 7 of a glucagon receptor.

In accordance with the present invention, the peptides comprise at least one amino acid that has a D-configuration. In one embodiment, the peptides comprise at least two amino acids that have a D-configuration. In another embodiment, the peptides comprise at least three amino acids that have a D-configuration. In a further embodiment, the peptides comprise at least four amino acids that have a D-configuration. In other embodiments, the peptides comprise at least five, at least six, and at least seven, amino acids that have a D-configuration.

In an alternate embodiment of the present invention, the peptides comprise all D-amino acids. In another alternate embodiment, the peptides are D-peptides that optionally comprise one or more L-amino acids.

The peptides of the present invention also include peptide derivatives that may further comprise one or more modifications and/or additional amino acids, which do not correspond to the sequence of the glucagon receptor. Such modifications can be at the N-terminus, the C-terminus, or both the N— and C-termini. Peptide derivatives may also comprise one or more modified amino acid within the peptide sequence, i.e. that is not at either the N— or the C-terminus. The presence of extra amino acids or modifications to one of the termini of the peptides may be desirable, for example, to improve the stability of the peptides, to incorporate a “tag” to aid in identification, detection or purification protocols, to improve solubility or to improve pharmokinetic parameters. By way of example, the solubility of the peptides may be improved by the addition of certain amino acids at the C-terminus. Examples of suitable amino acids that can be added at the C-terminal end to improve the solubility of the peptides include, but are not limited to, Lys, Gly-Lys and Gly-Lys-Lys. Other examples of modifications that can be made to the C-terminus of the peptide include, but are not limited to, amidation, and esterification.

Non-limiting examples of suitable modifications that may be made at the N-terminus include the addition of a R—CO— or a R—O—CO— group, wherein R is an alkyl, alkenyl, alkynyl, aryl, aralkyl, heteroalkyl, a heterocyclic ring, or a heteroaromatic ring. Said alkyl, alkenyl, alkynyl, aryl, aralkyl, heteroalkyl, heterocyclic ring, and heteroaromatic rings can be optionally substituted with one or more substituents independently selected from the group of alkyl, alkenyl, alkynyl, aryl, heteroalkyl, a heterocyclic ring, a heteroaromatic ring, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene and NZ₁Z₂ where Z₁ and Z₂ are independently hydrogen, alkyl, aryl, or aralkyl.

Non-limiting examples of suitable R—CO— groups are benzoyl, acetyl, t-butylacetyl, p-phenylbenzoyl, trifluoroacetyl, cyclohexylcarbonyl, phenylacetyl and 4-phenylbutanoyl, 3,3 diphenylpropanoyl, 4-biphenylacetyl, diphenylacetyl, 2-naphthylacetyl, 3-phenylbutanoyl, α-phenyl-ortho-toluoyl, indole-3-acetyl, 3-indolepropanoyl, 3-indolebutanoyl, 4-(4-methoxyphenyl)butanoyl, and the like.

Peptide derivatives further include cyclic peptides. A cyclic peptide can be produced through the formation of a peptide bond between the nitrogen atom at the N-terminus and the carbonyl carbon at the C-terminus. Alternatively, a cyclic peptide can be produced through formation of a covalent bond between the nitrogen at the N-terminus of the peptide and the side chain of a suitable amino acid within the peptide sequence. This can be the side chain of the C-terminal amino acid or an amino acid internal to the sequence. For example, an amide can be formed between the nitrogen atom at the N-terminus and the carbonyl carbon in the side chain of an aspartic acid or a glutamic acid. Cyclic peptides can also be produced by forming a covalent bond between the carbonyl at the C-terminus of the peptide and the side chain of a suitable amino acid in the peptide. This can be the side chain of the N-terminal amino acid or an amino acid internal to the sequence. For example, an amide can be formed between the carbonyl carbon at the C-terminus and the amino nitrogen atom in the side chain of a lysine, an ornithine, 2,3-diaminopropionic acid or 2,4-diaminobutyric acid. Additionally, cyclic peptides can be produced by forming an ester between the carbonyl carbon at the C-terminus and the hydroxyl oxygen atom in the side chain of a serine or a threonine within the peptide sequence.

Cyclic peptides can also be produced through the formation of a covalent bond between the side chains of two suitable amino acids within the peptide. These can be the side chains of the two terminal amino acids, the side chains of one terminal amino acid and one internal amino acid, or the side chains of two internal amino acids. For example, a disulphide bond can be formed between the sulphur atoms in the side chains of two cysteine residues, or an ester can be formed between the carbonyl carbon in the side chain of, for example, a glutamic acid or an aspartic acid, and the oxygen atom in the side chain of, for example, a serine or a threonine. Similarly, an amide can be formed between the carbonyl carbon in the side chain of, for example, a glutamic acid or an aspartic acid, and the amino nitrogen in side chain of, for example, a lysine, an ornithine, 2,3-diaminopropionic acid or 2,4-diaminobutyric acid. When necessary a peptide can be modified to include one or more appropriate naturally or non-naturally occurring amino acids to allow cyclisation.

In addition, cyclic peptides can be produced using a suitable linking group between the two terminal amino acids, between one terminal amino acid and the side chain of an internal amino acid, or between the side chains of two internal amino acids. Examples of suitable linking groups are known in the art and include those described in, for example, International Patent Applications WO 92/00995 and WO 94/15958.

Peptide derivatives also include tandem peptides in which a single amino acid sequence is repeated within the peptide. Alternatively, a tandem peptide may comprise two amino acid sequences, each corresponding wholly or in part to an extracellular membrane insertion region of a glucagon receptor, joined together.

The peptides of the invention can be a combination of peptide analogues, derivatives and/or variants. For example, in order to produce a cyclic peptide, a peptide of the invention can comprise an amino acid substitution to include a naturally or non-naturally occurring amino acid that comprises an appropriate side chain to allow cyclisation to occur. The cyclic peptide can further comprise one or more D-amino acid. Similarly, a peptide may comprise one or more non-naturally occurring amino acids and modification(s) at the N— and/or C-terminus and/or at one or more internal amino acids. Peptides can also comprise one or more substitution, deletion or insertion and modification(s) at the N— and/or C-terminus and/or at one or more internal amino acids. The peptides can further comprise one or more non-naturally occurring amino acids. Various other combinations are contemplated by the present invention and will be apparent to one skilled in the art.

In one embodiment of the present invention, the peptides comprise a sequence of three or more amino acids of a sequence selected from the group comprising: LVIDGLLRT (SEQ ID NO: 4); AAVRCGAV (SEQ ID NO: 5); FVTDEHAQ (SEQ ID NO: 6); QFSSYMKA (SEQ ID NO: 7); VVKCLPENV (SEQ ID NO:8); WFGMNDNS (SEQ ID NO:9) and FLKASRLT (SEQ ID NO:10). In another embodiment of the present invention, the peptides are a peptide analogue, peptide derivative, or a variant peptide of any one of SEQ ID NOs: 4, 5, 6, 7, 8, 9 or 10.

In a further embodiment of the invention, the peptides comprise one or more of the sequences provided in Table 2. In the Table, L-amino acids are represented by capital letters and D-amino acids are represented by small letters.

TABLE 2
Exemplary Peptide Sequences

| PEPTIDE SEQUENCE | SEQ ID NO |
|---|---|
| qfssymka | 65 |
| lvidgllrt | 66 |
| aavrcgav | 67 |
| vvkclpenv | 68 |
| wfgmndns | 69 |
| flkasrlt | 70 |
| dehaq | 71 |
| CfvtdehaqC | 72 |
| tdehaq | 73 |
| fvmdehaq | 74 |
| fvtdehar | 75 |
| fvmdehar | 76 |
| fitddqve | 77 |
| deHaq | 78 |
| fvtdehak | 79 |
| dehak | 80 |
| dehaK | 81 |
| fvtdehaqy | 82 |
| dehaqdehaq | 83 |
| dehaqy | 84 |
| dehadeha | 86 |

In another embodiment of the present invention, the peptides comprise a peptide derivative, peptide analogue or variant peptide, or a combination thereof of one or more amino acid sequences as set forth in SEQ ID NOS: 65-84 or 86. In an exemplary embodiment of the present invention, the peptides comprise a sequence as set forth in Table 3. In the Table, L-amino acids are represented by capital letters and D-amino acids are represented by small letters.

TABLE 3
Exemplary Peptides

| | |
|---|---|
| | SEQ |
| | ID |
| PEPTIDE SEQUENCE | NO |

| | |
|---|---|
| lvidgllrtGKK-COOH | 1 |
| aavrcgavGKK-COOH | 2 |
| fvtdehaqGKK-COOH | 3 |
| qfssymka-COOH | 11 |
| lvidgllrt-COOH | 12 |
| aavrcgav-COOH | 13 |
| vvkclpenv-COOH | 14 |
| w-f-g-m-n-d-n-s-COOH | 15 |
| f-l-k-a-s-r-l-t-COOH | 16 |
| q-f-s-s-y-m-k-a-G-K-K-COOH | 17 |
| v-v-k-c-l-p-e-n-v-G-K-K-COOH | 18 |
| w-f-g-m-n-d-n-s-G-K-K-COOH | 19 |
| f-l-k-a-s-r-l-t-G-K-K-COOH | 20 |
| l-v-i-d-g-l-l-r-t-G-K-COOH | 21 |
| a-a-v-r-c-g-a-v-G-K-COOH | 22 |
| f-v-t-d-e-h-a-q-G-K-COOH | 23 |
| f-l-k-a-s-r-l-t-G-K-COOH | 24 |
| f-v-t-d-e-h-a-a-G-K-COOH | 25 |
| f-v-t-d-e-h-G-K-COOH | 26 |
| 4-Biphenylacetyl-d-e-h-a-q-G-K-COOH | 27 |
| Diphenylacetyl-d-e-h-a-q-G-K-COOH | 28 |
| 2-Naphtylacetyl-d-e-h-a-q-G-K-COOH | 29 |
| 3-phenylbutanoyl-d-e-h-a-q-G-K-COOH | 30 |
| Benzenesulfonyl-β-alanine-d-e-h-a-q-G-K-COOH | 31 |
| α-Phenyl-O-toluoyl-dehaqGK-COOH | 32 |
| Indole-3-acetyl-d-e-h-a-q-G-K-COOH | 33 |
| 3-Indolepropanoyl-d-e-h-a-q-G-K-COOH | 34 |
| 3-Indolebutanoyl-d-e-h-a-q-G-K-COOH | 35 |
| Transcinnamic acid-d-e-h-a-q-G-K-COOH | 36 |
| C-f-v-t-d-e-h-a-q-C-G-K-COOH | 37 |
| 2-Naphtalene sulfonyl-β-alanine-dehaqGK-COOH | 38 |
| t-d-e-h-a-q-G-K-COOH | 39 |
| d-e-h-a-q-G-K-COOH | 40 |
| f-v-t-d-e-h-a-q-CONH₂ | 41 |
| f-v-m-d-e-h-a-q-G-K-COOH | 42 |
| f-v-t-d-e-h-a-r-G-K-COOH | 43 |
| f-v-m-d-e-h-a-r-G-K-COOH | 44 |
| f-i-t-d-d-q-v-e-G-K-COOH | 45 |
| 4-(4-Methoxyphenyl) butanoyl-d-e-H-a-q-G-K-COOH | 46 |
| f-v-t-d-e-h-a-q-COOH | 47 |
| f-v-t-d-e-h-a-k-CONH₂ | 48 |
| d-e-h-a-q-COOH | 49 |
| d-e-h-a-q-CONH₂ | 50 |
| d-e-h-a-k-CONH₂ | 51 |
| d-e-h-a-K-COOH | 52 |
| 3,3-diphenylpropanoyl-d-e-h-a-q-COOH | 53 |
| f-v-t-d-e-h-a-q-y-CONH2 | 54 |
| d-e-h-a-q-d-e-h-a-q-K-CONH₂ | 55 |
| d-e-h-a-q-y-CONH₂ | 56 |
| w-d-e-h-a-q-G-K-COOH | 57 |
| d-e-h-a-d-e-h-a-K-CONH₂ | 58 |
| Benzene sulfonyl-β-alanine-d-e-h-a-q-CONH₂ | 59 |

Other exemplary peptides include the cyclic peptides shown below:

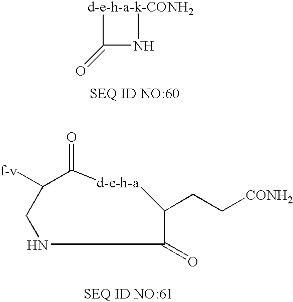

The chemical structure of SEQ ID NO:60 is as follows (all D-amino acids):

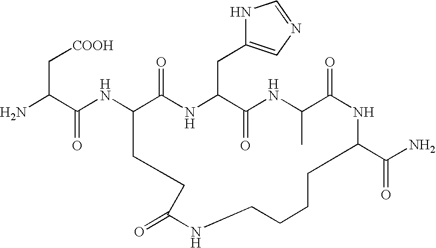

The chemical structure of SEQ ID NO:61 is as follows (all D-amino acids):

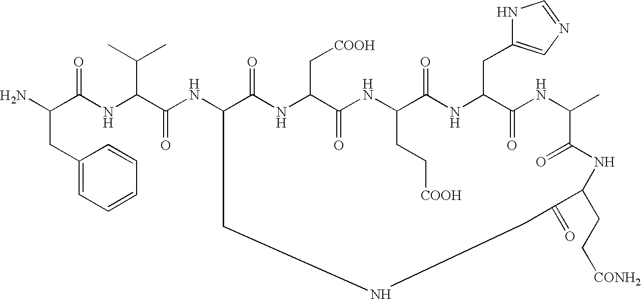

In another embodiment of the present invention, the peptides comprise a sequence as set forth in any one of SEQ ID NOs: 1, 2, 3, 23, 26, 27, 28, 29, 30, 31, 32, 33, 34, 35, 36, 37, 39, 40, 41, 42, 43, 44, 45, 46, 47, 48, 49, 50, 51, 52, 53, 55, 57, and 58. In a further embodiment, the peptides comprise an amino acid sequence as set forth in any one of SEQ ID NOS: 2, 23, 31, 40, 41, 47, 49, 50, 51, 53, 57, 59, 60, or 61. In a further embodiment, the peptides comprise the sequence: R—CO-dehaq or R—SO₂—β-alanine-dehaq, wherein R is aryl or aralkyl.

Preparation of the Peptides

The peptides of the present invention can be readily prepared by standard chemical synthesis techniques. The principles of solid phase chemical synthesis of polypeptides are well known in the art and may be found in general texts in the area such as Pennington, M. W. and Dunn, B. M., Methods in Molecular Biology, Vol. 35 (Humana Press, 1994); Dugas, H. and Penney, C., Bioorganic Chemistry (1981) Springer-Verlag, New York, pgs. 54-92; Merrifield, J. M., Chem. Soc., 85:2149 (1962), and Stewart and Young, Solid Phase Peptide Synthesis, pp. 24-66, Freeman (San Francisco, 1969).

Covalent modifications of the peptide can be introduced, for example, by reacting targeted amino acid residues with an organic derivatising agent that is capable of reacting with selected side chains or terminal residues as is known in the art. Selection of appropriate derivatising agent(s) can be readily accomplished by a worker skilled in the art.

The peptides of the present invention may also be prepared in their salt form. The peptides may be sufficiently acidic or sufficiently basic to react with a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like.

Base addition salts include those derived from inorganic bases, such as ammonium or alkali or alkaline earth metal hydroxides, carbonates, bicarbonates, and the like. Such bases useful in preparing the salts of this invention may be selected from the group of sodium hydroxide, potassium hydroxide, ammonium hydroxide, potassium carbonate, and the like.

A worker skilled in the art will readily understand that when the peptides in salt form are for therapeutic purposes, the salt will be a pharmaceutically acceptable salt.

Efficacy of the Peptides

The ability of the peptides of the present invention to modulate the glucagon response in a mammal can be determined in vitro or in vivo using standard techniques known in the art. For example, the ability of a candidate peptide to modulate cAMP levels in vitro or cAMP levels and/or blood glucose levels in an appropriate animal model can be determined. When an animal model is used, the effect of the candidate peptide on normal glucose levels can be measured, or the animal can be subjected to an appropriate treatment leading to increased or decreased blood glucose levels prior to administration of the peptide(s).

The candidate peptides can also be used in in vitro displacement studies such as those described herein (see Example 4).

An exemplary in vivo technique is provided below. One skilled in the art will appreciate that other similar tests may be conducted to determine the ability of the peptides to modulate the glucagon response in a mammal.

Appropriate amounts of the candidate peptide(s) are first administered to a group of normal animals (for example, rats or mice) by a suitable route, such as injection. Saline, or other suitable control, can be administered to a second group of animals, which acts as a control group. If desired, other control groups may be included to which known glucagon, or glucagon receptor, antagonists are administered. Typically, the animals have been fasted prior to the study. After an appropriate period of time (typically in the order of a few minutes), sufficient glucagon (or a glucagon agonist) is administered to the animals to provoke a glucagon response. Blood samples are drawn at appropriate time intervals after administration of the glucagon and are tested for blood glucose concentrations using standard techniques.

In accordance with one embodiment of the present invention, the peptides decrease the glucagon response. Thus, in one embodiment, the glucagon-induced increase in blood glucose levels in animals treated with the peptide is at least about 5% less than that in the control animals. In another embodiment, the glucagon-induced increase in blood glucose levels in animals treated with the peptide is at least about 10% less than that in the control animals. In another embodiment, the glucagon-induced increase is at least about 15% less than that in the control animals. Typically, the increase in blood glucose levels is measured 10, 15, 30, 45 or 60 minutes, or a combination thereof, after administration of glucagon.

Pharmaceutical Compositions

The peptides of the present invention may be formulated as pharmaceutical compositions with an appropriate pharmaceutically physiologically acceptable carrier, diluent, excipient or vehicle. The pharmaceutical compositions comprise one or more of the peptides and may further optionally comprise one or more other pharmaceutical compounds.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

The pharmaceutical compositions can be formulated in unit dosage form. The term “unit dosage form” refers to a physically discrete unit suitable as a unitary dosage for a mammal, such as a human, each unit containing a predetermined quantity of peptide calculated to produce the desired therapeutic effect in association with a suitable pharmaceutical excipient. For example, a suitable unit dosage form for the peptides of the invention may be one containing a dosage from about 10 μg to about 10 mg of each peptide.

The present invention also contemplates controlled release preparations. Such preparations usually comprise one or more polymer that serves to complex or absorb the peptide. Examples of such polymers include, but are not limited to, polyesters, polyamino acids, polyvinylpyrrolidone, ethylenevinyl acetate, methylcellulose, carboxymethylcellulose, and protamine sulfate, in an appropriate concentration and according to various methods of incorporation.

The duration of action of the peptide may also be controlled by incorporating the peptide into particles of a polymeric material. For example, particles comprising polyesters, polyamino acids, hydrogels, poly (lactic acid) or ethylene vinylacetate copolymers.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

Uses

The peptides of the present invention modulate the glucagon response in a mammal and, therefore, can be used to modulate cAMP levels and/or blood glucose levels in a mammal. The peptides have utility in the amelioration, treatment and/or prevention of glucagon-mediated diseases, disorders and conditions.

Examples of glucagon-mediated diseases, disorders and conditions that may be treated and/or prevented using one or more of the peptides of the invention include, for example, hyperglycemia, impaired glucose tolerance (IGT), insulin resistance syndromes, syndrome X, Type 1 diabetes, Type 2 diabetes, hyperlipidemia, dyslipidermia, hypertriglyceridemia, hyperlipoproteinemia, hypercholesterolemia, arteriosclerosis including atherosclerosis, glucagonomas, acute pancreatitis, cardiovascular disease, hypertension, cardiac hypertrophy, gastrointestinal disorders, obesity, diabetes as a consequence of obesity, and diabetic dylipidemia.

For treatment and/or prevention of Type 1 diabetes, the peptides may be used as part of a therapeutic regimen that includes insulin therapy. For treatment of diseases associated with obesity, the peptides may be used as part of a therapeutic regimen that includes diet and/or exercise modification.

The present invention thus provides methods of treating and/or preventing a glucagon-mediated disease, disorder and condition in a mammal comprising administering an effective amount of one or more of the peptides of the invention. In one embodiment, there is provided a method of treating and/or preventing hyperglycemia, impaired glucose tolerance (IGT), insulin resistance syndromes, Type 1 diabetes or Type 2 diabetes in a mammal comprising administering an effective amount of one or more of the peptides of the invention. In another embodiment, there is provided a method of treating and/or preventing a disease, disorder or condition associated with obesity in a mammal comprising administering an effective amount of one or more of the peptides of the invention.

Typical daily dosages to be administered are in the range from about 1 μg/kg to about 1 mg/kg of body weight, although lower or higher dosages may be administered. The dosage can be a single unit dose or it can be divided into sub-doses intended for administration over the course of the day. The required dosage will depend upon the severity of the condition of the subject and upon such criteria as the subject's height, weight, sex, age, and medical history and can readily be determined by one skilled in the art.

The present invention also contemplates the use of the peptides in combination with one or more other pharmaceutical agents in the treatment and/or prevention of the glucagon-mediated diseases, disorders or conditions. Examples of such pharmaceutical agents include antidiabetic agents, antihyperlipidemic agents, antiobesity agents, antihypertensive agents and agents for the treatment of complications resulting from or associated with diabetes.

Examples of suitable antidiabetic agents comprise insulin, insulin analogues and derivatives (such as N8B29-tetradecanoyl des (B30) human insulin, AspB28 human insulin, LysB28 ProB29 human insulin and Lantus), GLP-1 derivatives, orally active hypoglycaemic agents (such as imidazolines, sulphonylureas, biguanides, meglitinides, oxadiazolidinediones, thiazolidinediones, insulin sensitizers, glucosidase inhibitors, glucagon antagonists, GLP-1 agonists, agents acting on the ATP-dependent potassium channel of the a-cells, nateglinide or potassium channel blockers, insulin sensitizers, DPP-IV (dipeptidyl peptidase-IV) inhibitors, PTPase inhibitors, inhibitors of hepatic enzymes involved in stimulation of gluconeogenesis and/or glycogenolysis, glucose uptake modulators and GSK-3 (glycogen synthase kinase-3) inhibitors).

Examples of suitable antiobesity agents or appetite regulating agents include, but are not limited to, CART (cocaine amphetamine regulated transcript) agonists, NPY (neuropeptide Y) antagonists, MC4 (melanocortin 4) agonists, orexin antagonists, TNF (tumor necrosis factor) modulators, CRF (corticotropin releasing factor) agonists, CRF BP (corticotropin releasing factor binding protein) antagonists, urocortin agonists, 33 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline reuptake inhibitors, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators and TR 3 agonists.

Examples of suitable antihypertensive agents include, but are not limited to, 3-blockers (such as alprenolol, atenolol, timolol, pindolol, propranolol and metoprolol), ACE (angiotensin converting enzyme) inhibitors (such as benazepril, captopril, enalapril, fosinopril, lisinopril, quinapril and ramipril), calcium channel blockers (such as nifedipine, felodipine, nicardipine, isradipine, nimodipine, diltiazem and verapamil), α-blockers (such as doxazosin, prazosin and terazosin), and serotonin blockers (such as urapidil).

One or more of the peptides of the invention may be formulated into a pharmaceutical composition in combination with one or more other pharmaceutical agents for administration to a subject. Alternatively, the one or more peptide and the pharmaceutical agent(s) may be formulated separately. When separate formulations are used, they may be administered to the subject concurrently or they may be administered at different times.

Kits

The present invention additionally provides for therapeutic kits containing one or more peptides of the present invention, or a pharmaceutical composition comprising one or more peptide for use in the treatment of glucagon-mediated diseases, disorders or conditions. The kit may further comprise one or more other therapeutic agents to be used in combination with the peptide(s). Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or any such medically approved delivery vehicle.

To gain a better understanding of the invention described herein, the following examples are set forth. It should be understood that these examples are for illustrative purposes only. Therefore, they should not limit the scope of this invention in any way.

### EXAMPLES

### Example 1

### Synthesis of Peptides

The peptides of the present invention are prepared employing standard automated and/or manual solid phase peptide synthesis techniques (Pennington, M. W. and Dunn, B. M., Methods in Molecular Biology, Vol. 35 (Humana Press, 1994) using fluorenylmethoxycarbonyl-protected α-amino acids having appropriate side-chain protection. After completion of the synthesis, the peptide is cleaved from the solid phase support with simultaneous side-chain deprotection. Optionally, the corresponding acid of Aroyl or acyl was coupled to the N-terminus of the peptide using the same methods as was used for amino acid coupling. The crude peptides were further purified by preparative HPLC, followed by vacuum-drying and lyophilizing. The peptide purity was assessed by analytical HPLC and the peptide mass was determined by MALDI-TOF MS analysis or by any other mass spectrophotometry techniques known in the art. The peptides were prepared as TFA salts and dissolved in saline or 20 mM acetic acid for administration to animals.

The cyclised peptides were prepared using fluorenylmethoxycarbonyl-protected a-amino acids having allyl and alloc protection at the amino-acid side chains where the cyclisation will take place. As an example for peptide #60 the glutamic acid side chain is protected with allyl ester and the lysine ε-amine side chain is protected with alloc. At the end of the synthesis the allyl and alloc protecting group were removed using a palladium catalyst followed by cyclization using PyAOP and cleavage from the solid phase support with simultaneous side-chain deprotection. The crude peptides were further purified by preparative HPLC, followed by vacuum-drying and lyophilization. The peptide purity was assessed by analytical HPLC and the peptide mass was determined by MALDI-TOF MS analysis. Other mass spectrophotometry techniques known in the art can also be employed for this purpose.

A list of peptides prepared as described above are provided in Table 4. By convention, L-amino acids are represented by upper case letters and D-amino acids by lower case letters.

TABLE 4
Peptides

| | | |
|---|---|---|
| | SEQ | |
| Peptide | ID | |
| No. | NO | Peptide Sequence |

| | | |
|---|---|---|
| 1 | 1 | l-v-i-d-g-l-l-r-t-G-K-K-COOH |
| 2 | 2 | a-a-v-r-c-g-a-v-G-K-K-COOH |
| 3 | 3 | f-v-t-d-e-h-a-q-G-K-K-COOH |
| 11 | 11 | q-f-s-s-y-m-k-a-COOH |
| 12 | 12 | l-v-i-d-g-l-l-r-t-COOH |
| 13 | 13 | a-a-v-r-c-g-a-v-COOH |
| 14 | 14 | v-v-k-c-l-p-e-n-v-COOH |
| 15 | 15 | w-f-g-m-n-d-n-s-COOH |
| 16 | 16 | f-l-k-a-s-r-l-t-COOH |
| 17 | 17 | q-f-s-s-y-m-k-a-G-K-K-COOH |
| 18 | 18 | v-v-k-c-l-p-e-n-v-G-K-K-COOH |
| 19 | 19 | w-f-g-m-n-d-n-s-G-K-K-COOH |
| 20 | 20 | f-l-k-a-s-r-l-t-G-K-K-COOH |
| 21 | 21 | l-v-i-d-g-l-l-r-t-G-K-COOH |
| 22 | 22 | a-a-v-r-c-g-a-v-G-K-COOH |
| 23 | 23 | f-v-t-d-e-h-a-q-G-K-COOH |
| 24 | 24 | f-l-k-a-s-r-l-t-G-K-COOH |
| 25 | 25 | f-v-t-d-e-h-a-a-G-K-COOH |
| 26 | 26 | f-v-t-d-e-h-G-K-COOH |
| 27 | 27 | 4-Biphenylacetyl-d-e-h-a-q-G-K-COOH |
| 28 | 28 | Diphenylacetyl-d-e-h-a-q-G-K-COOH |
| 29 | 29 | 2-Naphtylacetyl-d-e-h-a-q-G-K-COOH |
| 30 | 30 | 3-phenylbutanoyl-d-e-h-a-q-G-K-COOH |
| 31 | 31 | Benzenesulfonyl-β-alanine-d-e-h-a-q- |
| | | G-K-COOH |
| 32 | 32 | α-Phenyl-O-toluoyl-d-e-h-a-q-G-K-COOH |
| 33 | 33 | Indole-3-acetyl-d-e-h-a-q-G-K-COOH |
| 34 | 34 | 3-Indolepropanoyl-d-e-h-a-q-G-K-COOH |
| 35 | 35 | 3-Indolebutanoyl-d-e-h-a-q-G-K-COOH |
| 36 | 36 | Transcinnamic acid-d-e-h-a-q-G-K-COOH |
| 37 | 37 | C-f-v-t-d-e-h-a-q-C-G-K-COOH |
| 38 | 38 | 2-Naphtalene sulfonyl-β-alanine-d-e- |
| | | h-a-q-G-K-COOH |
| 39 | 39 | t-d-e-h-a-q-G-K-COOH |
| 40 | 40 | d-e-h-a-q-G-K-COOH |
| 41 | 41 | f-v-t-d-e-h-a-q-CONH2 |
| 42 | 42 | f-v-m-d-e-h-a-q-G-K-COOH |
| 43 | 43 | f-v-t-d-e-h-a-r-G-K-COOH |
| 44 | 44 | f-v-m-d-e-h-a-r-G-K-COOH |
| 45 | 45 | f-i-t-d-d-q-v-e-G-K-COOH |
| 46 | 46 | 4-(4-Methoxyphenyl) butanoyl-d-e-H-a- |
| | | q-G-K-COOH |
| 47 | 47 | f-v-t-d-e-h-a-q-COOH |
| 48 | 48 | f-v-t-d-e-h-a-k-CONH2 |
| 49 | 49 | d-e-h-a-q-COOH |
| 50 | 50 | d-e-h-a-q-CONH2 |
| 51 | 51 | d-e-h-a-k-CONH2 |
| 52 | 52 | d-e-h-a-K-COOH |
| 53 | 53 | 3,3-diphenylpropanoyl-d-e-h-a-q-COOH |
| 54 | 54 | f-v-t-d-e-h-a-q-y-CONH2 |
| 55 | 55 | (d-e-h-a-q)₂-K-CONH2 |
| 56 | 56 | d-e-h-a-q-y-CONH2 |
| 57 | 57 | w-d-e-h-a-q-G-K-COOH |
| 58 | 58 | (d-e-h-a)₂-K-CONH2 |
| 59 | 59 | Benzene sulfonyl-β-alanine-d-e-h-a-q- |
| | | CONH2 |
| 62 | 62 | H-S-Q-G-T-F-T-S-D-Y-S-K-Y-L-D-S-R-R- |
| (native | | A-Q-D-F-V-Q-W-L-M-N-T-COOH |
| glucagon | | |
| amide) | | |
| 63 | 63 | S-Q-G-T-F-T-S-E-Y-S-K-Y-L-D-S-R-R-A- |
| [desHis1, | | Q-D-F-V-Q-W-L-M-N-T-CONH2 |
| Glu9] | | |
| glucagon | | |
| amide | | |
| 64 | 64 | H-A-E-G-T-F-T-S-D-V-S-S-Y-L-E-G-Q-A- |
| (GLP-1) | | A-K-E-F-I-A-W-L-V-K-G-R-NH2 |

The following peptides were also prepared:

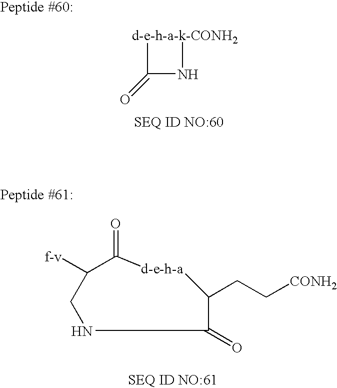

### Example 2

### Testing Peptides Nos. 1, 2 and 3 in a Rat Model of Glucagon-Induced Hyperglycemia

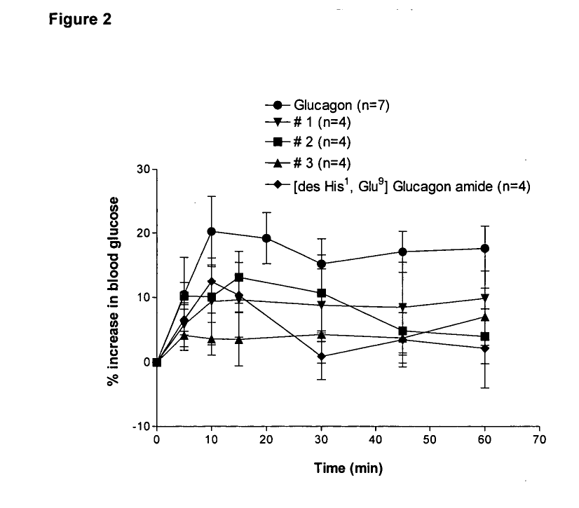

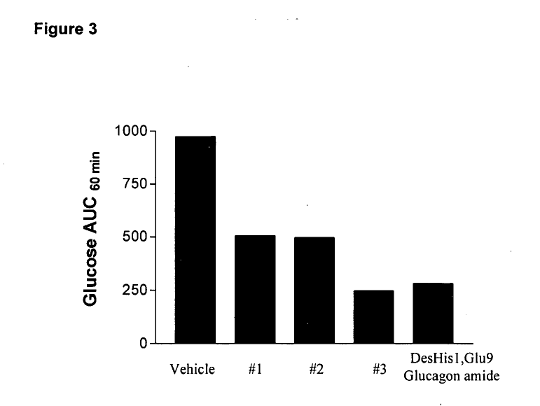

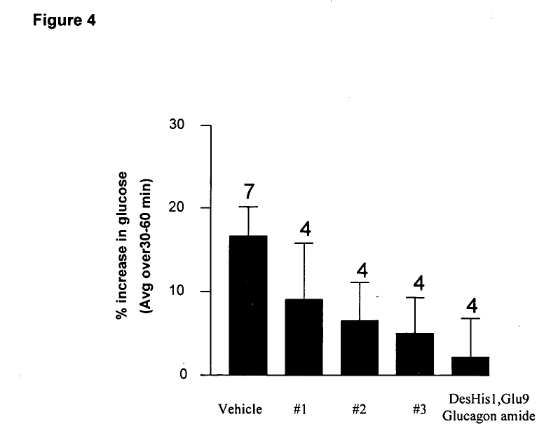

Normal male sprague-Dawley rats (290-320 g) fasted for 4-6 h were sedated with isofluorane and peptides were administered in saline via the jugular vein. Glucagon (1-29) amide (4 μg/rat), [des His¹], [Glu⁹] glucagon (1-29) amide (a known glucagon receptor antagonist) (10 μg/rat) and peptides nos. 1, 2 and 3 (1 mg/kg) were administered intravenously. The peptides and glucagon antagonist were administered 5 minutes prior to glucagon administration. Blood samples were drawn from the carotid artery at 0, 5, 10, 15, 30, 45 and 60-minute intervals and the glucose levels measured with a portable glucometer (Lifescan). The results are shown in FIGS. 2, 3 and 4.

### Example 3

### Effects of Peptides on cAMP Production in Isolated Hepatocytes

Hepatocytes Preparation

All experimental procedures were performed under isoflurane (2.5%) anesthesia according to an experimental protocol approved by the Ste-Justine animal care committee. Briefly, an incision is made across the abdomen of Sprague-Dawley rats to reveal the liver and to allow access to the superior vena cava. The animal is perfused through the heart to remove a maximal amount of blood from the liver (acquire a light brown color). A catheter (PE-90) is then inserted in the portal vein and the liver further perfused to eliminate any trace of blood. The hepatic artery is also cannulated (PE-50) and perfused. The liver is then carefully removed from the abdominal cavity and placed into a 250 ml beaker. Digesting HEPES buffer containing 9650U collagenase and 20U elastase at 37° C. is placed into the beaker and circulated in a closed loop via the catheters for 10 minutes at maximal speed. The buffer is replaced with a fresh solution of collagenase and elastase and perfusion is continued for 10 additional minutes. The liver is then transferred to a new beaker, to which buffer is added without collagenase or elastase and the hepatocytes dissociated by mechanical means (i.e. the peritoneum is opened and removed with scissors and tweezers and the liver agitated lightly for a few seconds) until pasty in appearance. The cells are filtrated with a tea strainer; the vascular tree and cell heaps remaining on the strainer. The cells are centrifuged at 52G for 3 minutes, resuspended and washed two more times. This gives approximately 120 to 160 million live cells from 1 liver (300 g rat).

cAMP Stimulation Assay

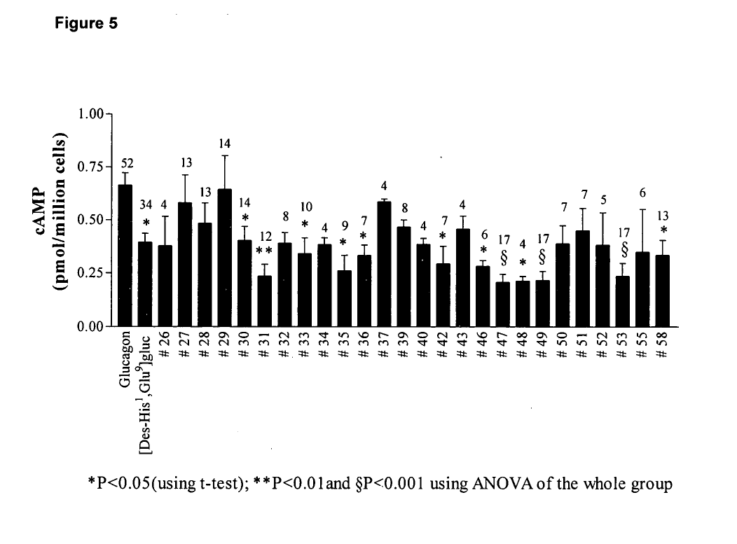

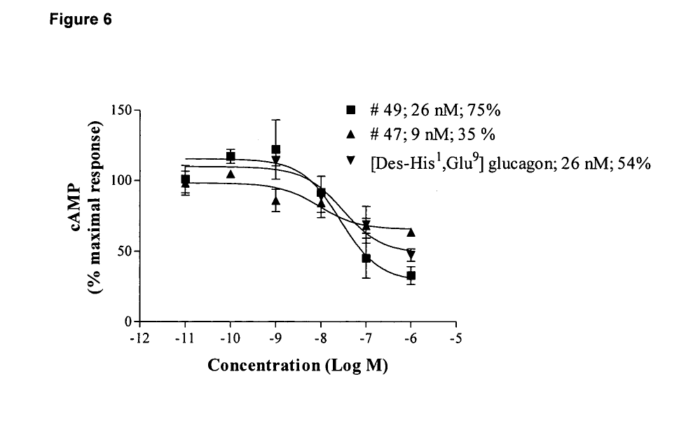

Stimulation studies are performed at a concentration of 1 million cells per tubes; 5 minutes of pretreatment with 0.1 mM IBMX (with or without the peptide (10⁻¹¹M to 10⁻⁶M)) followed by 5 minutes of treatment with glucagon (10⁻⁷M). Reactions are stopped on ice and stored at −80° C. prior to ETOH extraction. The cell pellets are thawed by adding 500 μl of 70% ETOH, vortexing the tubes for a few seconds and then incubating at 37° C. for 10 min. The tubes are centrifuged at 13,000×g for 10 min at 4° C. and the supernatants lyophilized in a speed-vac. The cAMP levels in the tubes were determined using a radioimmunoassay kit (Amersham DPC kit). Cells treated with Des [His 1] Glu9 glucagon amide (10⁻⁶ M) was used a control. The data are expressed as pmol cAMP/million cells. The results regarding various peptides are shown in FIGS. 5 and 6.

### Example 4

### Displacement Studies

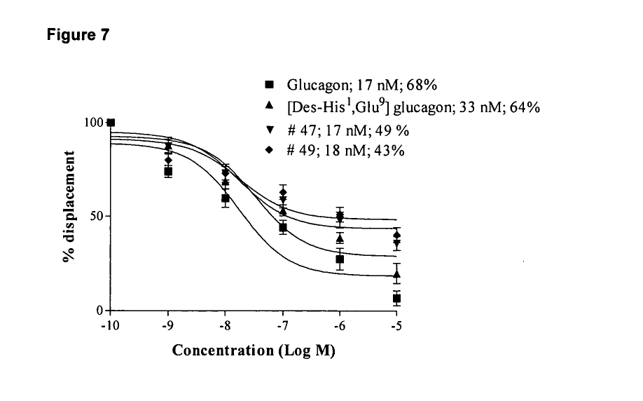

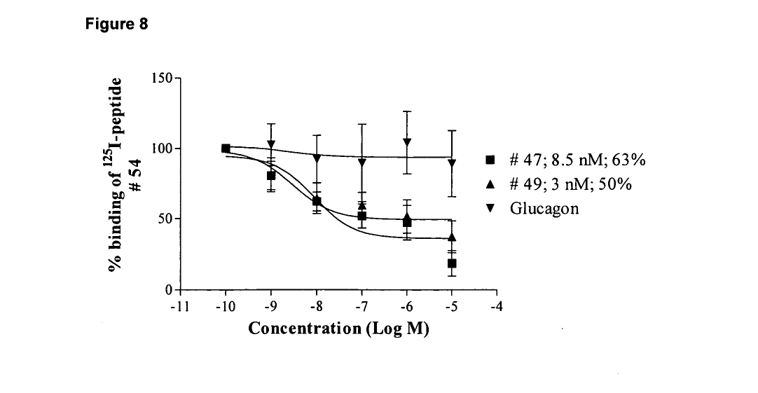

Prior to the displacement studies, filters (Whatman B) are soaked for a minimum of 1 hour in 5 mM Tris-HCl at room temperature. Serial dilutions of the test compounds (Peptide Nos: 47 and 49, glucagon amide (SEQ ID NO: 58) and/or [Des [His¹, Glu⁹] glucagon amide (Sigma, 81k49571) were prepared in incubation buffer (Phosphate buffer with protease inhibitor cocktail tablet (1 tablet/liter)) for a final concentrations in tubes of 10⁻¹⁰ to 10⁻⁵ M. The prepared peptide solutions (40 μl) are added to polystyrene tubes as well as the ¹²⁵I glucagon or ¹²⁵I Peptide No.54, (diluted to approximately 75000-150000 cpm in ddH2O, 10 μl). Hepatocytes (prepared in same manner described in Example 3) are then added in a timely fashion (50 μl of a 5 millions cells per ml, i.e. 12.5 mg/ml or 625 μg per tube) and incubated for 45 min at room temperature. The reaction is stopped with 100 mM Tris-HCl pH 7.4, and the mixture passed through the filters. The tubes are rinsed twice with 100 mM Tris-HCl pH 7.4 while the filter is rinsed once with the same solution. The vaccumed wet filters are then placed in tubes to be counted. The results are shown in FIGS. 7 and 8.

### Example 5

### In situ Liver Perfusion Assay

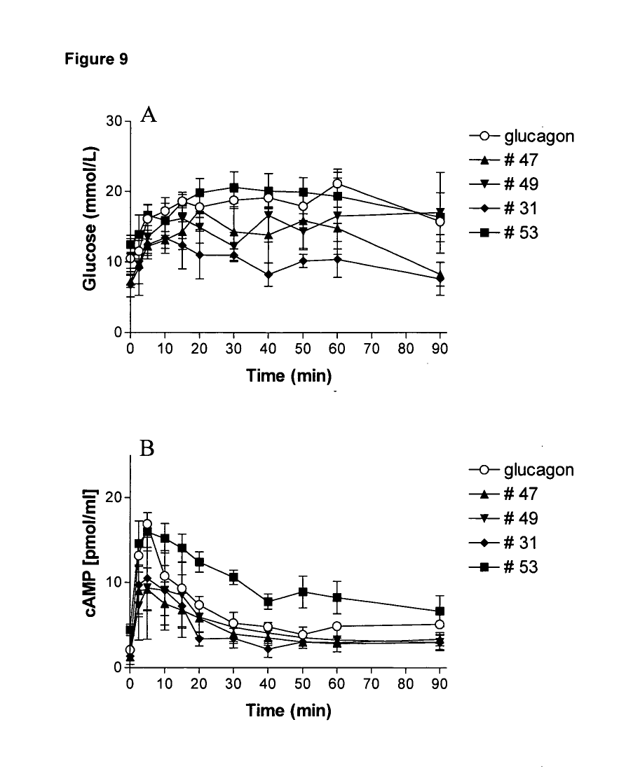

Sprague-Dawley rats are fasted 4 hours and then anesthetized under isoflurane (2.5%). An incision is then made across the abdomen to reveal the liver and allow access to the portal vein. A catheter (PE-90) is inserted in the portal vein (for drug injection) and in the carotid artery (for blood withdrawal). Prepared solutions of the peptides (Peptide Nos: 31, 47, 49, and 53) are injected subcutaneously (300 μg/kg) 10 minutes prior to the injection of glucagon (12 μg/kg) into the portal vein. Blood samples are taken at 0, 5, 10, 15, 20, 30, 40, 50, 60 and 90 minutes thereafter and blood glucose as well as cAMP levels measured. The results are shown in FIG. 9.

### Example 6

### Effects of Peptides on Blood Glucose Levels in Fasted CD-1 Mice

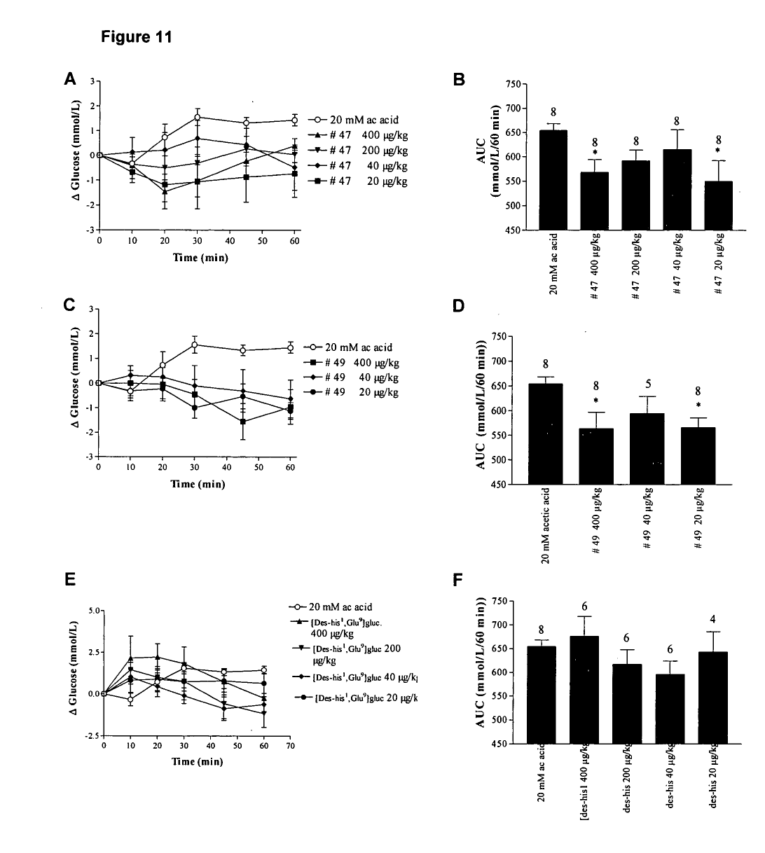

Five week old mice were placed in groups of four per cage where they were maintained on a 12:12 light:dark cycle and fed standard laboratory rodent chow. Water was provided ad libidum throughout the experimental period. One week later, after having fasted the mice for 4 hours, vehicle (20 mM acetic acid) or selected peptides (Peptide No: 47 or Peptide No: 49 (20, 40 or 400 μg/kg)) were given subcutaneously. Blood glucose levels were taken by interdigital punctures (mice were naive and not trained to be restrained for the interdigital punctures) prior to the injection and at various time intervals after the injection. The results are shown in FIG. 11.

### Example 7

### Effects of Peptides on Blood Glucose Levels in Fasted Sprague-Dawley Rats

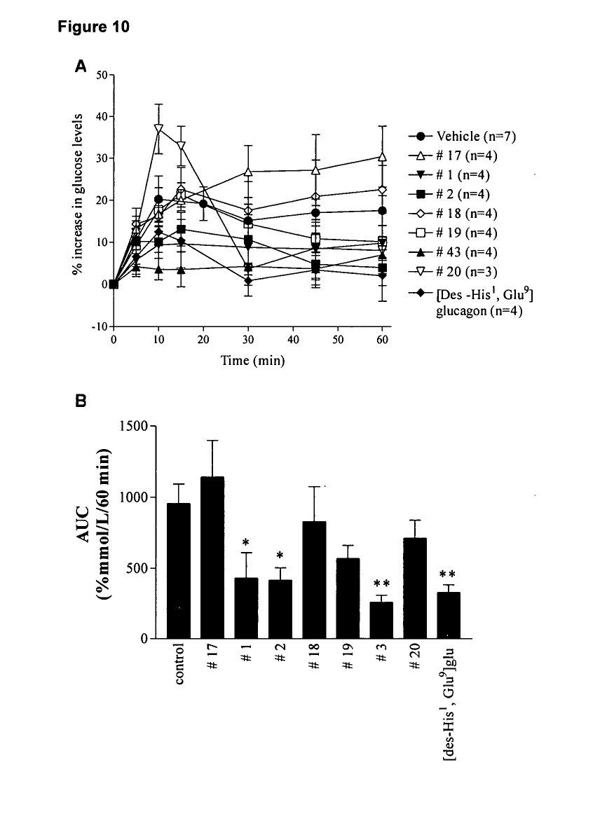

Sprague-Dawley rats weighing 250-300 g were received and placed in groups of four per cage, where they were maintained on a 12:12 light:dark cycle and fed standard laboratory rodent chow. Water was provided ad libidum throughout the experimental period. One week later, after having fasted the rats for 4 hours, vehicle (20 mM acetic acid) or selected peptides (Peptides No: 1, 2, 3, 17, 18, 19 and 20) (1 mg/Kg) were given subcutaneously. Blood glucose levels were taken by interdigital punctures (rats were naive and not trained to be restrained for the interdigital punctures) prior to the injection and at various time intervals after the injection. The results are shown in FIG. 10.

### Example 8

### Effects of Peptides on Blood Glucose Levels in STZ-Treated CD-1 Mice

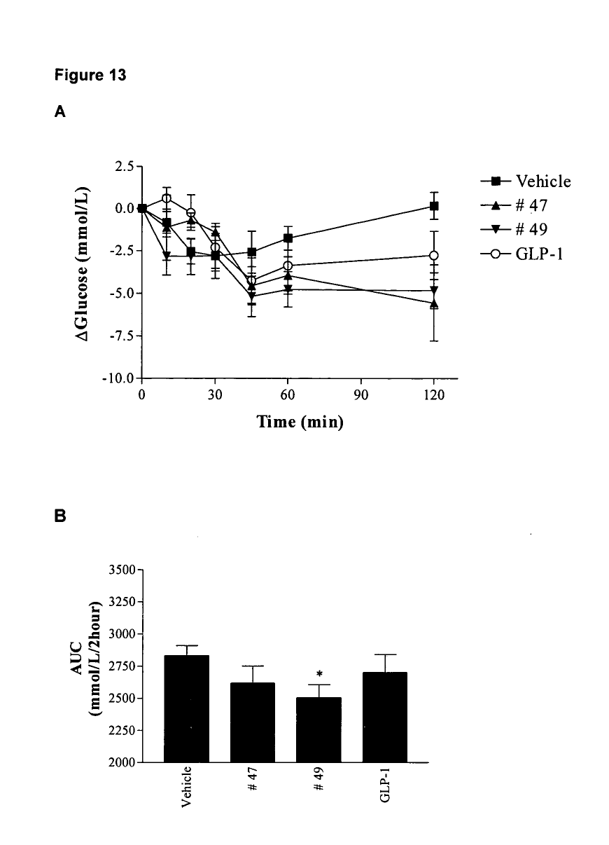

Five week old mice were placed in groups of four per cage, where they were maintained on a 12:12 light:dark cycle and fed standard laboratory rodent chow. Water was provided ad libidum throughout the experimental period. One week later, mice received an intraperitoneal injection of Streptozocin (STZ) at a dose of 400 mg/kg. Glycemia was markedly increased in the mice twenty-four (24) hours later (i.e. STZ treated 20-26 mmol/L; vehicle 10-12 mmol/L). At that time, either vehicle (20 mM acetic acid) or Peptide Nos. 47 or 49 (400 μg/kg) was given subcutaneously and blood glucose levels were measured prior to and following the injection at various time intervals. Glucose measurements were made using a drop of blood obtained from a tail cut and a portable glucometer (Accucheck compact, Roche). The results are shown in FIG. 13.

### Example 9

### Effects of Peptides on Stress-Induced Blood Glucose Levels in Sprague-Dawley Rats

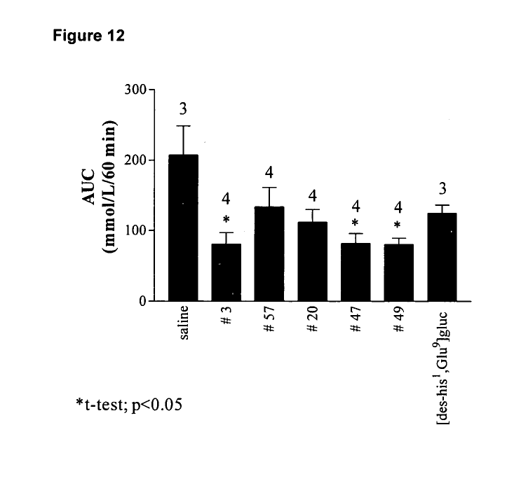

Sprague-Dawley rats weighing 250-300 g were placed in groups of four per cage, where they were maintained on a 12:12 light:dark cycle and fed standard laboratory rodent chow. Water was provided ad libidum throughout the experimental period. One week later, the rats were fasted four hours, and the effects of the peptides were assessed on stress-induced glucose increase in Sprague-Dawley rats. Saline or peptide nos. 3, 24, 47, 49 and 57 were given subcutaneously (300 μg/kg). Blood glucose measurements were taken at 60 minutes post injection using a drop of blood obtained from an interdigital puncture and a portable glucometer (Accuchek compact, Roche). The results are shown in FIG. 12.

### Example 10

### Effects of Peptides on Stress-Induced Blood Glucose Levels in Diabetic Rats

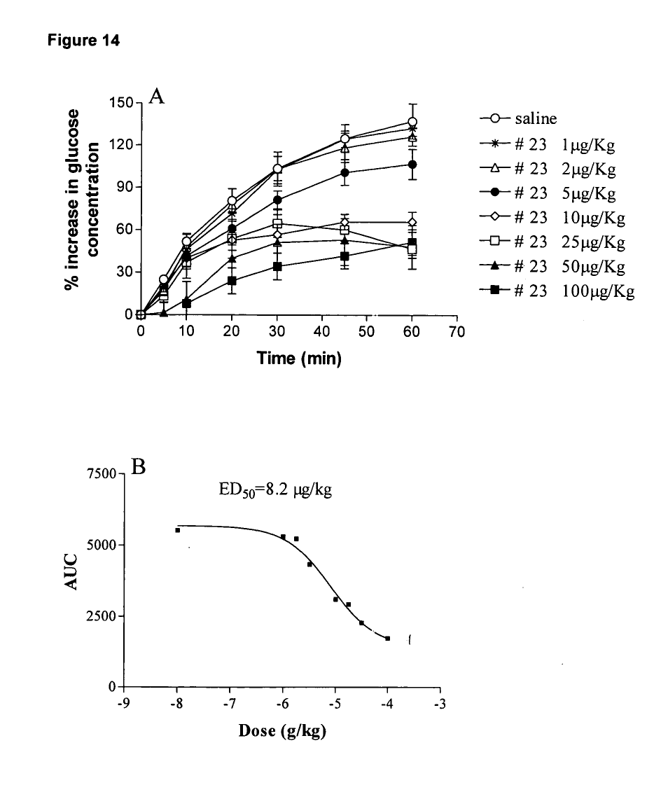

Diabetic fa/fa rats weighing 800-900 g were kept in groups of two per cage where they were maintained on a 12:12 light:dark cycle and fed standard laboratory rodent chow. Water was provided ad libidum throughout the experimental period. The dose-dependent effects of Peptide No. 23 (1, 2, 5, 10, 25, 50, and 100 μg/kg) was assessed on the stress-induced glucose increase in the fa/fa rats. Blood samples were taken prior to peptide administration and at various time intervals (0, 5, 10, 30, 45, 60 minutes) after the peptide administration (rats were restrained and blood samples taken by interdigital puncture). The results are shown in FIG. 14.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A peptide comprising an amino acid between about 5 and about 10 amino acids in length, said amino acid sequence corresponding to a sequence of an extracellular membrane insertion region of a mammalian glucagon receptor and comprising at least one D-amino acid, wherein said peptide is capable of modulating the glucagon response in a mammal.

2. The peptide according to claim 1, wherein said amino acid sequence is between about 7 and about 9 amino acids in length.

3. The peptide according to claim 1, wherein said peptide is a peptide analogue, peptide derivative, variant peptide, peptidomimetic or a combination thereof.

4. The peptide according to claim 2, wherein said peptide comprises all D-amino acids.

5. The peptide according to claim 2, wherein said peptide comprises a C-terminal modification.

6. The peptide according to claim 5, wherein said C-terminal modification is selected from the group of: an amidation and addition of one or more amino acids.

7. The peptide according to claim 2, wherein said peptide comprises a N-terminal modification.

8. The peptide according to claim 7, wherein said N-terminal modification comprises addition of a R—CO— or a R—O—CO— group, wherein R is an alkyl, alkenyl, alkynyl, aryl, aralkyl, heteroalkyl, a heterocyclic ring, or a heteroaromatic ring and wherein said alkyl, alkenyl, alkynyl, aryl, aralkyl, heteroalkyl, heterocyclic ring, and heteroaromatic rings is optionally substituted with one or more substituents independently selected from the group of: alkyl, alkenyl, alkynyl, aryl, heteroalkyl, a heterocyclic ring, a heteroaromatic ring, aralkyl, hydroxy, alkoxy, aralkyloxy, aryloxy, carboxy, acyl, aroyl, halo, nitro, trihalomethyl, cyano, alkoxycarbonyl, aryloxycarbonyl, aralkoxycarbonyl, acylamino, aroylamino, dialkylamino, carbamoyl, alkylcarbamoyl, dialkylcarbamoyl, alkylthio, aralkylthio, arylthio, alkylene and NZ₁Z₂ where Z₁ and Z₂ are independently hydrogen, alkyl, aryl, and aralkyl.

9. The peptide according to claim 1, wherein said peptide comprises both a N-terminal modification and a C-terminal modification.

10. The peptide according to claim 1, wherein said peptide comprises a sequence as set forth in any one of SEQ ID NOs:65-84.

11. The peptide according to claim 1, wherein said peptide has the sequence R—CO-dehaq, wherein R is aryl or an aralkyl.

12. The peptide according to claim 1, wherein said peptide has the sequence R—SO2-β-alanine-dehaq, wherein R is aryl or an aralkyl.

13. The peptide according to claim 1, wherein said peptide comprises an amino acid sequence as set forth in any one of SEQ ID NOs: 1, 2, 3, 26, 27, 28, 29, 30, 31, 32, 33, 34, 35, 36, 37, 39, 40, 41, 42, 43, 44, 45, 46, 47, 48, 49, 50, 51, 52, 53, 55, 57 and 58.

14. The peptide according to claim 1, wherein the peptide comprises an amino acid sequence as set forth in any one of SEQ ID NOs: 2, 23, 31, 40, 41, 47, 49, 50, 51, 53, 59, 60, 61.

15. A pharmaceutical composition comprising one or more peptide according to claim 1 and a pharmaceutically acceptable diluent, carrier or excipient.

16. A method of modulating cAMP levels in a mammal comprising administering to said mammal an effective amount of one or more peptide according to claim 1.

17. A method of modulating blood glucose levels in a mammal comprising administering to said mammal an effective amount of one or more peptide according to claim 1.

18. A method of modulating the glucagon response in a mammal comprising administering to said mammal an effective amount of one or more peptide according to claim 1.

19. A method of treating or preventing a glucagon-mediated disease, disorder or condition in a mammal comprising administering to said mammal an effective amount of one or more peptide according to claim 1.

20. The method according to claim 19, wherein said glucagon-mediated disease, disorder or condition is selected from the group of: diabetes, hyperglycemia, impaired glucose tolerance (IGT), insulin resistance syndromes, syndrome X, hyperlipidemia, dyslipidermia, hypertriglyceridemia, hyperlipoproteinemia, hypercholesterolemia, arteriosclerosis, glucagonomas, acute pancreatitis, cardiovascular disease, hypertension, cardiac hypertrophy, gastrointestinal disorders, and diabetic dylipidemia.

21. The method according to claim 19, wherein said glucagon-mediated disease, disorder or condition is Type 1 diabetes, Type 2 diabetes or hyperglycemia.

22. The method according to claim 19, wherein said glucagon-mediated disease, disorder or condition is associated with obesity.

23. A method of determining the ability of a peptide to modulate the glucagon response, said method comprising the steps of:
d) contacting cells or tissue responsive to glucagon with a candidate peptide and a known glucagon antagonist;
e) after an appropriate period of time, contacting said cells or tissue with glucagon to elicit a glucagon response; and
f) measuring one or more biochemical consequences of the cell before the addition of glucagon and at appropriate time intervals after the addition of glucagon, wherein said biochemical consequence is cAMP or glucose levels;
wherein a change in the measured biochemical consequence compared to a negative control indicates that said peptide is capable of modulating the glucagon response.

24. A kit comprising:
c) one or more peptides according to claim 1; and
d) optionally instructions for use.
